SEQUENCE LISTING (3 sequences)

SEQ ID NO: 1 - protein, 352 aa, Homo sapiens [sequence omitted]

SEQ ID NO: 2 - DNA, 29903 bp, SARS coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 3 - DNA, 30119 bp, MERS coronavirus
gatttaagtgaatagcttggctatctcacttcccctcgttctcttgcagaactttgattttaacgaacttaaataaaagccctgttgtttagcgtatcgttgcacttgtctggtgggattgtggcattaatttgcctgctcatctaggcagtggacatatgctcaacactgggtataattctaattgaatactatttttcagttagagcgtcgtgtctcttgtacgtctcggtcacaatacacggtttcgtccggtgcgtggcaattcggggcacatcatgtctttcgtggctggtgtgaccgcgcaaggtgcgcgcggtacgtatcgagcagcgctcaactctgaaaaacatcaagaccatgtgtctctaactgtgccactctgtggttcaggaaacctggttgaaaaactttcaccatggttcatggatggcgaaaatgcctatgaagtggtgaaggccatgttacttaaaaaggagccacttctctatgtgcccatccggctggctggacacactagacacctcccaggtcctcgtgtgtacctggttgagaggctcattgcttgtgaaaatccattcatggttaaccaattggcttatagctctagtgcaaatggcagcctggttggcacaactttgcagggcaagcctattggtatgttcttcccttatgacatcgaacttgtcacaggaaagcaaaatattctcctgcgcaagtatggccgtggtggttatcactacaccccattccactatgagcgagacaacacctcttgccctgagtggatggacgattttgaggcggatcctaaaggcaaatatgcccagaatctgcttaagaagttgattggcggtgatgtcactccagttgaccaatacatgtgtggcgttgatggaaaacccattagtgcctacgcatttttaatggccaaggatggaataaccaaactggctgatgttgaagcggacgtcgcagcacgtgctgatgacgaaggcttcatcacattaaagaacaatctatatagattggtttggcatgttgagcgtaaagacgttccatatcctaagcaatctatttttactattaatagtgtggtccaaaaggatggtgttgaaaacactcctcctcactattttactcttggatgcaaaattttaacgctcaccccacgcaacaagtggagtggcgtttctgacttgtccctcaaacaaaaactcctttacaccttctatggtaaggagtcacttgagaacccaacctacatttaccactccgcattcattgagtgtggaagttgtggtaatgattcctggcttacagggaatgctatccaagggtttgcctgtggatgtggggcatcatatacagctaatgatgtcgaagtccaatcatctggcatgattaagccaaatgctcttctttgtgctacttgcccctttgctaagggtgatagctgttcttctaattgcaaacattcagttgctcagttggttagttacctttctgaacgctgtaatgttattgctgattctaagtccttcacacttatctttggtggcgtagcttacgcctactttggatgtgaggaaggtactatgtactttgtgcctagagctaagtctgttgtctcaaggattggagactccatctttacaggctgtactggctcttggaacaaggtcactcaaattgctaacatgttcttggaacagactcagcattcccttaactttgtgggagagttcgttgtcaacgatgttgtcctcgcaattctctctggaaccacaactaatgttgacaaaatacgccagcttctcaaaggtgtcacccttgacaagttgcgtgattatttagctgactatgacgtagcagtcactgccggcccattcatggataatgctattaatgttggtggtacaggattacagtatgccgccattactgcaccttatgtagttctcactggcttaggtgagtcctttaagaaagttgcaaccataccgtataaggtttgcaactctgttaaggatactctggcttattatgctcacagcgtgttgtacagagtttttccttatgacatggattctggtgtgtcatcctttagtgaactactttttgattgcgttgatctttcagtagcttctacctattttttagtccgcatcttgcaagataagactggcgactttatgtctacaattattacttcctgccaaactgctgttagtaagcttctagatacatgttttgaagctacagaagcaacatttaacttcttgttagatttggcaggattgttcagaatctttctccgcaatgcctatgtgtacacttcacaagggtttgtggtggtcaatggcaaagtttctacacttgtcaaacaagtgttagacttgcttaataagggtatgcaacttttgcatacaaaggtctcctgggctggttctaaaatcattgctgttatctacagcggcagggagtctctaatattcccatcgggaacctattactgtgtcaccactaaggctaagtccgttcaacaagatcttgacgttattttgcctggtgagttttccaagaagcagttaggactgctccaacctactgacaattctacaactgttagtgttactgtatccagtaacatggttgaaactgttgtgggtcaacttgagcaaactaatatgcatagtcctgatgttatagtaggtgactatgtcattattagtgaaaaattgtttgtgcgtagtaaggaagaagacggatttgccttctaccctgcttgcactaatggtcatgctgtaccgactctctttagacttaagggaggtgcacctgtaaaaaaagtagcctttggcggtgatcaagtacatgaggttgctgctgtaagaagtgttactgtcgagtacaacattcatgctgtattagacacactacttgcttcttctagtcttagaacctttgttgtagataagtctttgtcaattgaggagtttgctgacgtagtaaaggaacaagtctcagacttgcttgttaaattactgcgtggaatgccgattccagattttgatttagacgattttattgacgcaccatgctattgctttaacgctgagggtgatgcatcctggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgagacttctgacgacgagtgggctgctgcagttgatgaagcgttccctctcgatgaagcagaagatgttactgaatctgtgcaagaagaagcacaaccagtagaagtacctgttgaagatattgcgcaggttgtcatagctgacaccttacaggaaactcctgttgtgcctgatactgttgaagtcccaccgcaagtggtgaaacttccgtctgcacctcagactatccagcccgaggtaaaagaagttgcacctgtctatgaggctgataccgaacagacacagaatgttactgttaaacctaagaggttacgcaaaaagcgtaatgttgaccctttgtccaattttgaacataaggttattacagagtgcgttaccatagttttaggtgacgcaattcaagtagccaagtgctatggggagtctgtgttagttaatgctgctaacacacatcttaagcatggcggtggtatcgctggtgctattaatgcggcttcaaaaggggctgtccaaaaagagtcagatgagtatattctggctaaagggccgttacaagtaggagattcagttctcttgcaaggccattctctagctaagaatatcctgcatgtcgtaggcccagatgcccgcgctaaacaggatgtttctctccttagtaagtgctataaggctatgaatgcatatcctcttgtagtcactcctcttgtttcagcaggcatatttggtgtaaaaccagctgtgtcttttgattatcttattagggaggctaagactagagttttagtcgtcgttaattcccaagatgtctataagagtcttaccatagttgacattccacagagtttgactttttcatatgatgggttacgtggcgcaatacgtaaagctaaagattatggttttactgtttttgtgtgcacagacaactctgctaacactaaagttcttaggaacaagggtgttgattatactaagaagtttcttacagttgacggtgtgcaatattattgctacacgtctaaggacactttagatgatatcttacaacaggctaataagtctgttggtattatatctatgcctttgggatatgtgtctcatggtttagacttaatgcaagcagggagtgtcgtgcgtagagttaacgtgccctacgtgtgtctcctagctaataaagagcaagaagctattttgatgtctgaagacgttaagttaaacccttcagaagattttataaagcacgtccgcactaatggtggttacaattcttggcatttagtcgagggtgaactattggtgcaagacttacgcttaaataagctcctgcattggtctgatcaaaccatatgctacaaggatagtgtgttttatgttgtaaagaatagtacagcttttccatttgaaacactttcagcatgtcgtgcgtatttggattcacgcacgacacagcagttaacaatcgaagtcttagtgactgtcgatggtgtaaattttagaacagtcgttctaaataataagaacacttatagatcacagcttggatgcgttttctttaatggtgctgatatttctgacaccattcctgatgagaaacagaatggtcacagtttatatctagcagacaatttgactgctgatgaaacaaaggcgcttaaagagttatatggccccgttgatcctactttcttacacagattctattcacttaaggctgcagtccatgggtggaagatggttgtgtgtgataaggtacgttctctcaaattgagtgataataattgttatcttaatgcagttattatgacacttgatttattgaaggacattaaatttgttatacctgctctacagcatgcatttatgaaacataagggcggtgattcaactgacttcatagccctcattatggcttatggcaattgcacatttggtgctccagatgatgcctctcggttacttcataccgtgcttgcaaaggctgagttatgctgttctgcacgcatggtttggagagagtggtgcaatgtctgtggcataaaagatgttgttctacaaggcttaaaagcttgttgttacgtgggtgtgcaaactgttgaagatctgcgtgctcgcatgacatatgtatgccagtgtggtggtgaacgtcatcggcaattagtcgaacacaccaccccctggttgctgctctcaggcacaccaaatgaaaaattggtgacaacctccacggcgcctgattttgtagcatttaatgtctttcagggcattgaaacggctgttggccattatgttcatgctcgcctgaagggtggtcttattttaaagtttgactctggcaccgttagcaagacttcagactggaagtgcaaggtgacagatgtacttttccccggccaaaaatacagtagcgattgtaatgtcgtacggtattctttggacggtaatttcagaacagaggttgatcccgacctatctgctttctatgttaaggatggtaaatactttacaagtgaaccacccgtaacatattcaccagctacaattttagctggtagtgtctacactaatagctgccttgtatcgtctgatggacaacctggcggtgatgctattagtttgagttttaataaccttttagggtttgattctagtaaaccagtcactaagaaatacacttactccttcttgcctaaagaagacggcgatgtgttgttggctgagtttgacacttatgaccctatttataagaatggtgccatgtataaaggcaaaccaattctttgggtcaataaagcatcttatgatactaatcttaataagttcaatagagctagtttgcgtcaaatttttgacgtagcccccattgaactcgaaaataaattcacacctttgagtgtggagtctacaccagttgaacctccaactgtagatgtggtagcacttcaacaggaaatgacaattgtcaaatgtaagggtttaaataaacctttcgtgaaggacaatgtcagtttcgttgctgatgattcaggtactcccgttgttgagtatctgtctaaagaagacctacatacattgtatgtagaccctaagtatcaagtcattgtcttaaaagacaatgtactttcttctatgcttagattgcacaccgttgagtcaggtgatattaacgttgttgcagcttccggatctttgacacgtaaagtgaagttactatttagggcttcattttatttcaaagaatttgctacccgcactttcactgctaccactgctgtaggtagttgtataaagagtgtagtgcggcatctaggtgttactaaaggcatattgacaggctgttttagttttgccaagatgttatttatgcttccactagcttactttagtgattcaaaactcggcaccacagaggttaaagtgagtgctttgaaaacagccggcgttgtgacaggtaatgttgtaaaacagtgttgcactgctgctgttgatttaagtatggataagttgcgccgtgtggattggaaatcaaccctacggttgttacttatgttatgcacaactatggtattgttgtcttctgtgtatcacttgtatgtcttcaatcaggtcttatcaagtgatgttatgtttgaagatgcccaaggtttgaaaaagttctacaaagaagttagagcttacctaggaatctcttctgcttgtgacggtcttgcttcagcttatagggcgaattcctttgatgtacctacattctgcgcaaaccgttctgcaatgtgtaattggtgcttgattagccaagattccataactcactacccagctcttaagatggttcaaacacatcttagccactatgttcttaacatagattggttgtggtttgcatttgagactggtttggcatacatgctctatacctcggccttcaactggttgttgttggcaggtacattgcattatttctttgcacagacttccatatttgtagactggcggtcatacaattatgctgtgtctagtgccttctggttattcacccacattccaatggcgggtttggtacgaatgtataatttgttagcatgcctttggcttttacgcaagttttatcagcatgtaatcaatggttgcaaagatacggcatgcttgctctgctataagaggaaccgacttactagagttgaagcttctaccgttgtctgtggtggaaaacgtacgttttatatcacagcaaatggcggtatttcattctgtcgtaggcataattggaattgtgtggattgtgacactgcaggtgtggggaataccttcatctgtgaagaagtcgcaaatgacctcactaccgccctacgcaggcctattaacgctacggatagatcacattattatgtggattccgttacagttaaagagactgttgttcagtttaattatcgtagagacggtcaaccattctacgagcggtttcccctctgcgcttttacaaatctagataagttgaagttcaaagaggtctgtaaaactactactggtatacctgaatacaactttatcatctacgactcatcagatcgtggccaggaaagtttagctaggtctgcatgtgtttattattctcaagtcttgtgtaaatcaattcttttggttgactcaagtttggttacttctgttggtgattctagtgaaatcgccactaaaatgtttgattcctttgttaatagtttcgtctcgctgtataatgtcacacgcgataagttggaaaaacttatctctactgctcgtgatggcgtaaggcgaggcgataacttccatagtgtcttaacaacattcattgacgcagcacgaggccccgcaggtgtggagtctgatgttgagaccaatgaaattgttgactctgtgcagtatgctcataaacatgacatacaaattactaatgagagctacaataattatgtaccctcatatgttaaacctgatagtgtgtctaccagcgatttaggtagtctcattgattgtaatgcggcttcagttaaccaaattgtcttgcgtaattctaatggtgcttgcatttggaacgctgctgcatatatgaaactctcggatgcacttaaacgacagattcgcattgcatgccgtaagtgtaatttagctttccggttaaccacctcaaagctacgcgctaatgataatatcttatcagttagattcactgctaacaaaattgttggtggtgctcctacatggtttaatgcgttgcgtgactttacgttaaagggttatgttcttgctaccattattgtgtttctgtgtgctgtactgatgtatttgtgtttacctacattttctatggcacctgttgaattttatgaagaccgcatcttggactttaaagttcttgataatggtatcattagggatgtaaatcctgatgataagtgctttgctaataagcaccggtccttcacacaatggtatcatgagcatgttggtggtgtctatgacaactctatcacatgcccattgacagttgcagtaattgctggagttgctggtgctcgcattccagacgtacctactacattggcttgggtgaacaatcagataattttctttgtttctcgagtctttgctaatacaggcagtgtttgctacactcctatagatgagataccctataagagtttctctgatagtggttgcattcttccatctgagtgcactatgtttagggatgcagagggccgtatgacaccatactgccatgatcctactgttttgcctggggcttttgcgtacagtcagatgaggcctcatgttcgttacgacttgtatgatggtaacatgtttattaaatttcctgaagtagtatttgaaagtacacttaggattactagaactctgtcaactcagtactgccggttcggtagttgtgagtatgcacaagagggtgtttgtattaccacaaatggctcgtgggccatttttaatgaccaccatcttaatagacctggtgtctattgtggctctgattttattgacattgtcaggcggttagcagtatcactgttccagcctattacttatttccaattgactacctcattggtcttgggtataggtttgtgtgcgttcctgactttgctcttctattatattaataaagtaaaacgtgcttttgcagattacacccagtgtgctgtaattgctgttgttgctgctgttcttaatagcttgtgcatctgctttgttacctctataccattgtgtatagtaccttacactgcattgtactattatgctacattctattttactaatgagcctgcatttattatgcatgtttcttggtacattatgttcgggcctatcgttcccatatggatgacctgcgtctatacagttgcaatgtgctttagacacttcttctgggttttagcttattttagtaagaaacatgtagaagtttttactgatggtaagcttaattgtagtttccaggacgctgcctctaatatctttgttattaacaaggacacttatgcagctcttagaaactctttaactaatgatgcctattcacgatttttggggttgtttaacaagtataagtacttctctggtgctatggaaacagccgcttatcgtgaagctgcagcatgtcatcttgctaaagccttacaaacatacagcgagactggtagtgatcttctttaccaaccacccaactgtagcataacctctggcgtgttgcaaagcggtttggtgaaaatgtcacatcccagtggagatgttgaggcttgtatggttcaggttacctgcggtagcatgactcttaatggtctttggcttgacaacacagtctggtgcccacgacacgtaatgtgcccggctgaccagttgtctgatcctaattatgatgccttgttgatttctatgactaatcatagtttcagtgtgcaaaaacacattggcgctccagcaaacttgcgtgttgttggtcatgccatgcaaggcactcttttgaagttgactgtcgatgttgctaaccctagcactccagcctacacttttacaacagtgaaacctggcgcagcatttagtgtgttagcatgctataatggtcgtccgactggtacattcactgttgtaatgcgccctaactacacaattaagggttcctttctgtgtggttcttgtggtagtgttggttacaccaaggagggtagtgtgatcaatttctgttacatgcatcaaatggaacttgctaatggtacacataccggttcagcatttgatggtactatgtatggtgcctttatggataaacaagtgcaccaagttcagttaacagacaaatactgcagtgttaatgtagtagcttggctttacgcagcaatacttaatggttgcgcttggtttgtaaaacctaatcgcactagtgttgtttcttttaatgaatgggctcttgccaaccaattcactgaatttgttggcactcaatccgttgacatgttagctgtcaaaacaggcgttgctattgaacagctgctttatgcgatccaacaactgtatactgggttccagggaaagcaaatccttggcagtaccatgttggaagatgaattcacacctgaggatgttaatatgcagattatgggtgtggttatgcagagtggtgtgagaaaagttacatatggtactgcgcattggttgtttgcgacccttgtctcaacctatgtgataatcttacaagccactaaatttactttgtggaactacttgtttgagactattcccacacagttgttcccactcttatttgtgactatggccttcgttatgttgttggttaaacacaaacacacctttttgacacttttcttgttgcctgtggctatttgtttgacttatgcaaacatagtctacgagcccactactcccatttcgtcagcgctgattgcagttgcaaattggcttgcccccactaatgcttatatgcgcactacacatactgatattggtgtctacattagtatgtcacttgtattagtcattgtagtgaagagattgtacaacccatcactttctaactttgcgttagcattgtgcagtggtgtaatgtggttgtacacttatagcattggagaagcctcaagccccattgcctatctggtttttgtcactacactcactagtgattatacgattacagtctttgttactgtcaaccttgcaaaagtttgcacttatgccatctttgcttactcaccacagcttacacttgtgtttccggaagtgaagatgatacttttattatacacatgtttaggtttcatgtgtacttgctattttggtgtcttctctcttttgaaccttaagcttagagcacctatgggtgtctatgactttaaggtctcaacacaagagttcagattcatgactgctaacaatctaactgcacctagaaattcttgggaggctatggctctgaactttaagttaataggtattggcggtacaccttgtataaaggttgctgctatgcagtctaaacttacagatcttaaatgcacatctgtggttctcctctctgtgctccaacagttacacttagaggctaatagtagggcctgggctttctgtgttaaatgccataatgatatattggcagcaacagaccccagtgaggctttcgagaaattcgtaagtctctttgctactttaatgactttttctggtaatgtagatcttgatgcgttagctagtgatatttttgacactcctagcgtacttcaagctactctttctgagttttcacacttagctacctttgctgagttggaagctgcgcagaaagcctatcaggaagctatggactctggtgacacctcaccacaagttcttaaggctttgcagaaggctgttaatatagctaaaaacgcctatgagaaggataaggcagtggcccgtaagttagaacgtatggctgatcaggctatgacttctatgtataagcaagcacgtgctgaagacaagaaagcaaaaattgtcagtgctatgcaaactatgttgtttggtatgattaagaagctcgacaacgatgttcttaatggtatcatttctaacgctaggaatggttgtatacctcttagtgtcatcccactgtgtgcttcaaataaacttcgcgttgtaattcctgacttcaccgtctggaatcaggtagtcacatatccctcgcttaactacgctggggctttgtgggacattacagttataaacaatgtggacaatgaaattgttaagtcttcagatgttgtagacagcaatgaaaatttaacatggccacttgttttagaatgcactagggcatccacttctgccgttaagttgcaaaataatgagatcaaaccttcaggtctaaaaaccatggttgtgtctgcgggtcaagagcaaactaactgtaatactagttccttagcttattacgaacctgtgcagggtcgtaaaatgctgatggctcttctttctgataatgcctatctcaaatgggcgcgtgttgaaggtaaggacggatttgtcagtgtagagctacaacctccttgcaaattcttgattgcgggaccaaaaggacctgaaatccgatatctctattttgttaaaaatcttaacaaccttcatcgcgggcaagtgttagggcacattgctgcgactgttagattgcaagctggttctaacaccgagtttgcctctaattcctcggtgttgtcacttgttaacttcaccgttgatcctcaaaaagcttatctcgatttcgtcaatgcgggaggtgccccattgacaaattgtgttaagatgcttactcctaaaactggtacaggtatagctatatctgttaaaccagagagtacagctgatcaagagacttatggtggagcttcagtgtgtctctattgccgtgcgcatatagaacatcctgatgtctctggtgtttgtaaatataagggtaagtttgtccaaatccctgctcagtgtgtccgtgaccctgtgggattttgtttgtcaaataccccctgtaatgtctgtcaatattggattggatatgggtgcaattgtgactcgcttaggcaagcagcactgccccaatctaaagattccaattttttaaacgagtccggggttctattgtaaatgcccgaatagaaccctgttcaagtggtttgtccactgatgtcgtctttagggcatttgacatctgcaactataaggctaaggttgctggtattggaaaatactacaagactaatacttgtaggtttgtagaattagatgaccaagggcatcatttagactcctattttgtcgttaagaggcatactatggagaattatgaactagagaagcactgttacgacttgttacgtgactgtgatgctgtagctccccatgatttcttcatctttgatgtagacaaagttaaaacacctcatattgtacgtcagcgtttaactgagtacactatgatggatcttgtatatgccctgaggcactttgatcaaaatagcgaagtgcttaaggctatcttagtgaagtatggttgctgtgatgttacctactttgaaaataaactctggtttgattttgttgaaaatcccagtgttattggtgtttatcataaacttggagaacgtgtacgccaagctatcttaaacactgttaaattttgtgaccacatggtcaaggctggtttagtcggtgtgctcacactagacaaccaggaccttaatggcaagtggtatgattttggtgacttcgtaatcactcaacctggttcaggagtagctatagttgatagctactattcttatttgatgcctgtgctctcaatgaccgattgtctggccgctgagacacatagggattgtgattttaataaaccactcattgagtggccacttactgagtatgattttactgattataaggtacaactctttgagaagtactttaaatattgggatcagacgtatcacgcaaattgcgttaattgtactgatgaccgttgtgtgttacattgtgctaatttcaatgtattgtttgctatgaccatgcctaagacttgtttcggacccatagtccgaaagatctttgttgatggcgtgccatttgtagtatcttgtggttatcactacaaagaattaggtttagtcatgaatatggatgttagtctccatagacataggctctctcttaaggagttgatgatgtatgccgctgatccagccatgcacattgcctcctctaacgcttttcttgatttgaggacatcatgttttagtgtcgctgcacttacaactggtttgacttttcaaactgtgcggcctggcaattttaaccaagacttctatgatttcgtggtatctaaaggtttctttaaggagggctcttcagtgacgctcaaacattttttctttgctcaagatggtaatgctgctattacagattataattactattcttataatctgcctactatgtgtgacatcaaacaaatgttgttctgcatggaagttgtaaacaagtacttcgaaatctatgacggtggttgtcttaatgcttctgaagtggttgttaataatttagacaagagtgctggccatccttttaataagtttggcaaagctcgtgtctattatgagagcatgtcttaccaggagcaagatgaactttttgccatgacaaagcgtaacgtcattcctaccatgactcaaatgaatctaaaatatgctattagtgctaagaatagagctcgcactgttgcaggcgtgtccatacttagcacaatgactaatcgccagtaccatcagaaaatgcttaagtccatggctgcaactcgtggagcgacttgcgtcattggtactacaaagttctacggtggctgggatttcatgcttaaaacattgtacaaagatgttgataatccgcatcttatgggttgggattaccctaagtgtgatagagctatgcctaatatgtgtagaatcttcgcttcactcatattagctcgtaaacatggcacttgttgtactacaagggacagattttatcgcttggcaaatgagtgtgctcaggtgctaagcgaatatgttctatgtggtggtggttactacgtcaaacctggaggtaccagtagcggagatgccaccactgcatatgccaatagtgtctttaacattttgcaggcgacaactgctaatgtcagtgcacttatgggtgctaatggcaacaagattgttgacaaagaagttaaagacatgcagtttgatttgtatgtcaatgtttacaggagcactagcccagaccccaaatttgttgataaatactatgcttttcttaataagcacttttctatgatgatactgtctgatgacggtgtcgtttgctataatagtgattatgcagctaagggttacattgctggaatacagaattttaaggaaacgctgtattatcagaacaatgtctttatgtctgaagctaaatgctgggtggaaaccgatctgaagaaagggccacatgaattctgttcacagcatacgctttatattaaggatggcgacgatggttacttccttccttatccagacccttcaagaattttgtctgccggttgctttgtagatgatatcgttaagactgacggtacactcatggtagagcggtttgtgtctttggctatagatgcttaccctctcacaaagcatgaagatatagaataccagaatgtattctgggtctacttacagtatatagaaaaactgtataaagaccttacaggacacatgcttgacagttattctgtcatgctatgtggtgataattctgctaagttttgggaagaggcattctatagagatctctatagttcgcctaccactttgcaggctgtcggttcatgcgttgtatgccattcacagacttccctacgctgtgggacatgcatccgtagaccatttctctgctgtaaatgctgctatgatcatgttatagcaactccacataagatggttttgtctgtttctccttacgtttgtaatgcccctggttgtggcgtttcagacgttactaagctatatttaggtggtatgagctacttttgtgtagatcatagacctgtgtgtagttttccactttgcgctaatggtcttgtattcggcttatacaagaatatgtgcacaggtagtccttctatagttgaatttaataggttggctacctgtgactggactgaaagtggtgattacacccttgccaatactacaacagaaccactcaaactttttgctgctgagactttacgtgccactgaagaggcgtctaagcagtcttatgctattgccaccatcaaagaaattgttggtgagcgccaactattacttgtgtgggaggctggcaagtccaaaccaccactcaatcgtaattatgtttttactggttatcatataaccaaaaatagtaaagtgcagctcggtgagtacattttcgagcgcattgattatagtgatgctgtatcctacaagtctagtacaacgtataaactgactgtaggtgacatcttcgtacttacctctcactctgtggctaccttgacggcgcccacaattgtgaatcaagagaggtatgttaaaattactgggttgtacccaaccattacggtacctgaagagttcgcaagtcatgttgccaacttccaaaaatcaggttatagtaaatatgtcactgttcagggaccacctggcactggcaaaagtcattttgctatagggttagcgatttactaccctacagcacgtgttgtttatacagcatgttcacacgcagctgttgatgctttgtgtgaaaaagcttttaaatatttgaacattgctaaatgttcccgtatcattcctgcaaaggcacgtgttgagtgctatgacaggtttaaagttaatgagacaaattctcaatatttgtttagtactattaatgctctaccagaaacttctgccgatattctggtggttgatgaggttagtatgtgcactaattatgatctttcaattattaatgcacgtattaaagctaagcacattgtctatgtaggagatccagcacagttgccagctcctaggactttgttgactagaggcacattggaaccagaaaatttcaatagtgtcactagattgatgtgtaacttaggtcctgacatatttttaagtatgtgctacaggtgtcctaaggaaatagtaagcactgtgagcgctcttgtctacaataataaattgttagccaagaaggagctttcaggccagtgctttaaaatactctataagggcaatgtgacgcatgatgctagctctgccattaatagaccacaactcacatttgtgaagaattttattactgccaatccggcatggagtaaggcagtctttatttcgccttacaattcacagaatgctgtgtctcgttcaatgctgggtcttaccactcagactgttgattcctcacagggttcagaataccagtacgttatcttctgtcaaacagcagatacggcacatgctaacaacattaacagatttaatgttgcaatcactcgtgcccaaaaaggtattctttgtgttatgacatctcaggcactctttgagtccttagagtttactgaattgtcttttactaattacaagctccagtctcagattgtaactggcctttttaaagattgctctagagaaacttctggcctctcacctgcttatgcaccaacatatgttagtgttgatgacaagtataagacgagtgatgagctttgcgtgaatcttaatttacccgcaaatgtcccatactctcgtgttatttccaggatgggctttaaactcgatgcaacagttcctggatatcctaagcttttcattactcgtgaagaggctgtaaggcaagttcgaagctggataggcttcgatgttgagggtgctcatgcttcccgtaatgcatgtggcaccaatgtgcctctacaattaggattttcaactggtgtgaactttgttgttcagccagttggtgttgtagacactgagtggggtaacatgttaacgggcattgctgcacgtcctccaccaggtgaacagtttaagcacctcgtgcctcttatgcataagggggctgcgtggcctattgttagacgacgtatagtgcaaatgttgtcagacactttagacaaattgtctgattactgtacgtttgtttgttgggctcatggctttgaattaacgtctgcatcatacttttgcaagataggtaaggaacagaagtgttgcatgtgcaatagacgcgctgcagcgtactcttcacctctgcaatcttatgcctgctggactcattcctgcggttatgattatgtctacaaccctttctttgtcgatgttcaacagtggggttatgtaggcaatcttgctactaatcacgatcgttattgctctgtccatcaaggagctcatgtggcttctaatgatgcaataatgactcgttgtttagctattcattcttgttttatagaacgtgtggattgggatatagagtatccttatatctcacatgaaaagaaattgaattcctgttgtagaatcgttgagcgcaacgtcgtacgtgctgctcttcttgccggttcatttgacaaagtctatgatattggcaatcctaaaggaattcctattgttgatgaccctgtggttgattggcattattttgatgcacagcccttgaccaggaaggtacaacagcttttctatacagaggacatggcctcaagatttgctgatgggctctgcttattttggaactgtaatgtaccaaaatatcctaataatgcaattgtatgcaggtttgacacacgtgtgcattctgagttcaatttgccaggttgtgatggcggtagtttgtatgttaacaagcacgcttttcatacaccagcatatgatgtgagtgcattccgtgatctgaaacctttaccattcttttattattctactacaccatgtgaagtgcatggtaatggtagtatgatagaggatattgattatgtacccctaaaatctgcagtctgtattacagcttgtaatttagggggcgctgtttgtaggaagcatgctacagagtacagagagtatatggaagcatataatcttgtctctgcatcaggtttccgcctttggtgttataagacctttgatatttataatctctggtctacttttacaaaagttcaaggtttggaaaacattgcttttaatgttgttaaacaaggccattttattggtgttgagggtgaactacctgtagctgtagtcaatgataagatcttcaccaagagtggcgttaatgacatttgtatgtttgagaataaaaccactttgcctactaatatagcttttgaactctatgctaagcgtgctgtacgctcgcatcccgatttcaaattgctacacaatttacaagcagacatttgctacaagttcgtcctttgggattatgaacgtagcaatatttatggtactgctactattggtgtatgtaagtacactgatattgatgttaattcagctttgaatatatgttttgacatacgcgataattgttcattggagaagttcatgtctactcccaatgccatctttatttctgatagaaaaatcaagaaatacccttgtatggtaggtcctgattatgcttacttcaatggtgctatcatccgtgatagtgatgttgttaaacaaccagtgaagttctacttgtataagaaagtcaataatgagtttattgatcctactgagtgtatttacactcagagtcgctcttgtagtgacttcctacccctttctgacatggagaaagactttctatcttttgatagtgatgttttcattaagaagtatggcttggaaaactatgcttttgagcacgtagtctatggagacttctctcatactacgttaggcggtcttcacttgcttattggtttatacaagaagcaacaggaaggtcatattattatggaagaaatgctaaaaggtagctcaactattcataactattttattactgagactaacacagcggcttttaaggcggtgtgttctgttatagatttaaagcttgacgactttgttatgattttaaagagtcaagaccttggcgtagtatccaaggttgtcaaggttcctattgacttaacaatgattgagtttatgttatggtgtaaggatggacaggttcaaaccttctaccctcgactccaggcttctgcagattggaaacctggtcatgcaatgccatccctctttaaagttcaaaatgtaaaccttgaacgttgtgagcttgctaattacaagcaatctattcctatgcctcgcggtgtgcacatgaacatcgctaaatatatgcaattgtgccagtatttaaatacttgcacattagccgtgcctgccaatatgcgtgttatacattttggcgctggttctgataaaggtatcgctcctggtacctcagttttacgacagtggcttcctacagatgccattattatagataatgatttaaatgagttcgtgtcagatgctgacataactttatttggagattgtgtaactgtacgtgtcggccaacaagtggatcttgttatttccgacatgtatgatcctactactaagaatgtaacaggtagtaatgagtcaaaggctttattctttacttacctgtgtaacctcattaataataatcttgctcttggtgggtctgttgctattaaaataacagaacactcttggagcgttgaactttatgaacttatgggaaaatttgcttggtggactgttttctgcaccaatgcaaatgcatcctcatctgaaggattcctcttaggtattaattacttgggtactattaaagaaaatatagatggtggtgctatgcacgccaactatatattttggagaaattccactcctatgaatctgagtacttactcactttttgatttatccaagtttcaattaaaattaaaaggaacaccagttcttcaattaaaggagagtcaaattaacgaactcgtaatatctctcctgtcgcagggtaagttacttatccgtgacaatgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaattaacgaactattaatgagagttcaaagaccacccactctcttgttagtgttttcactctctcttttggtcactgcatcctcaaaacctctctatgtacctgagcattgtcagaattattctggttgcatgcttagggcttgtattaaaactgcccaagctgatacagctggtctttatacaaattttcgaattgacgtcccatctgcagaatcaactggtactcaatcagtttctgtcgatcttgagtcaacttcaactcatgatggtcctaccgaacatgttactagtgtgaatctttttgacgttggttactcagttaattaacgaactctatggattacgtgtctctgcttaatcaaatttggcagaagtaccttaactcaccgtatactacttgtttgtacatccctaaacccacagctaagtatacacctttagttggcacttcattgcaccctgtgctgtggaactgtcagctatcctttgctggttatactgaatctgctgttaattctacaaaagctttggccaaacaggacgcagctcagcgaatcgcttggttgctacataaggatggaggaatccctgatggatgttccctctacctccggcactcaagtttattcgcgcaaagcgaggaagaggagccattctccaactaagaaactgcgctacgttaagcgtagattttctcttctgcgccatgaagaccttagtgttattgtccaaccaacacactatgtcagggttacattttcagaccccaacatgtggtatctacgttcgggtcatcatttacactcagttcacaattggcttaaaccttatggcggccaacctgtttctgagtaccatattactctagctttgctaaatctcactgatgaagatttagctagagatttttcacccattgcgctctttttgcgcaatgtcagatttgagctacatgagttcgccttgctgcgcaaaactcttgttcttaatgcatcagagatctactgtgctaacatacatagatttaagcctgtgtatagagttaacacggcaatccctactattaaggattggcttctcgttcagggattttccctttaccatagtggcctccctttacatatgtcaatctctaaattgcatgcactggatgatgttactcgcaattacatcattacaatgccatgctttagaacttaccctcaacaaatgtttgttactcctttggccgtagatgttgtctccatacggtcttccaatcagggtaataaacaaattgttcattcttatcccattttacatcatccaggattttaacgaactatggctttctcggcgtctttatttaaacccgtccagctagtcccagtttctcctgcatttcatcgcattgagtctactgactctattgttttcacatacattcctgctagcggctatgtagctgctttagctgtcaatgtgtgtctcattcccctattattactgctacgtcaagatacttgtcgtcgcagcattatcagaactatggttctctatttccttgttctgtataactttttattagccattgtactagtcaatggtgtacattatccaactggaagttgcctgatagccttcttagttatcctcataatactttggtttgtagatagaattcgtttctgtctcatgctgaattcctacattccactgtttgacatgcgttcccactttattcgtgttagtacagtttcttctcatggtatggtccctgtaatacacaccaaaccattatttattagaaacttcgatcagcgttgcagctgttctcgttgtttttatttgcactcttccacttatatagagtgcacttatattagccgttttagtaagattagcctagtttctgtaactgacttctccttaaacggcaatgtttccactgttttcgtgcctgcaacgcgcgattcagttcctcttcacataatcgccccgagctcgcttatcgtttaagcagctctgcgctactatgggtcccgtgtagaggctaatccattagtctctctttggacatatggaaaacgaactatgttaccctttgtccaagaacgaatagggttgttcatagtaaactttttcatttttaccgtagtatgtgctataacactcttggtgtgtatggctttccttacggctactagattatgtgtgcaatgtatgacaggcttcaataccctgttagttcagcccgcattatacttgtataatactggacgttcagtctatgtaaaattccaggatagtaaaccccctctaccacctgacgagtgggtttaacgaactccttcataatgtctaatatgacgcaactcactgaggcgcagattattgccattattaaagactggaactttgcatggtccctgatctttctcttaattactatcgtactacagtatggatacccatcccgtagtatgactgtctatgtctttaaaatgtttgttttatggctcctatggccatcttccatggcgctatcaatatttagcgccgtttatccaattgatctagcttcccagataatctctggcattgtagcagctgtttcagctatgatgtggatttcctactttgtgcagagtatccggctgtttatgagaactggatcatggtggtcattcaatcctgagactaattgccttttgaacgttccatttggtggtacaactgtcgtacgtccactcgtagaggactctaccagtgtaactgctgttgtaaccaatggccacctcaaaatggctggcatgcatttcggtgcttgtgactacgacagacttcctaatgaagtcaccgtggccaaacccaatgtgctgattgctttaaaaatggtgaagcggcaaagctacggaactaattccggcgttgccatttaccatagatataaggcaggtaattacaggagtccgcctattacggcggatattgaacttgcattgcttcgagcttaggctctttagtaagagtatcttaattgattttaacgaatctcaatttcattgttatggcatcccctgctgcacctcgtgctgtttcctttgccgataacaatgatataacaaatacaaacctatctcgaggtagaggacgtaatccaaaaccacgagctgcaccaaataacactgtctcttggtacactgggcttacccaacacgggaaagtccctcttacctttccacctgggcagggtgtacctcttaatgccaattctacccctgcgcaaaatgctgggtattggcggagacaggacagaaaaattaataccgggaatggaattaagcaactggctcccaggtggtacttctactacactggaactggacccgaagcagcactcccattccgggctgttaaggatggcatcgtttgggtccatgaagatggcgccactgatgctccttcaacttttgggacgcggaaccctaacaatgattcagctattgttacacaattcgcgcccggtactaagcttcctaaaaacttccacattgaggggactggaggcaatagtcaatcatcttcaagagcctctagcttaagcagaaactcttccagatctagttcacaaggttcaagatcaggaaactctacccgcggcacttctccaggtccatctggaatcggagcagtaggaggtgatctactttaccttgatcttctgaacagactacaagcccttgagtctggcaaagtaaagcaatcgcagccaaaagtaatcactaagaaagatgctgctgctgctaaaaataagatgcgccacaagcgcacttccaccaaaagtttcaacatggtgcaagcttttggtcttcgcggaccaggagacctccagggaaactttggtgatcttcaattgaataaactcggcactgaggacccacgttggccccaaattgctgagcttgctcctacagccagtgcttttatgggtatgtcgcaatttaaacttacccatcagaacaatgatgatcatggcaaccctgtgtacttccttcggtacagtggagccattaaacttgacccaaagaatcccaactacaataagtggttggagcttcttgagcaaaatattgatgcctacaaaaccttccctaagaaggaaaagaaacaaaaggcaccaaaagaagaatcaacagaccaaatgtctgaacctccaaaggagcagcgtgtgcaaggtagcatcactcagcgcactcgcacccgtccaagtgttcagcctggtccaatgattgatgttaacactgattagtgtcactcaaagtaacaagatcgcggcaatcgtttgtgtttggcaaccccatctcaccatcgcttgtccactcttgcacagaatggaatcatgttgtaattacagtgcaataaggtaattataacccatttaattgatagctatgctttattaaagtgtgtagctgtagagagaatgttaaagactgtcacctctgcttgattgcaagtgaacagtgccccccgggaagagctctacagtgtgaaatgtaaataaaaaatagctattattcaattagattaggctaattagatgatttgcaaaaaaaaaaaa

(19) United States
(12) Patent Application Publication
(10) Pub. No.: US 2023/0172912 A1
(43) Pub. Date: Jun. 8, 2023

(54) COMPOUNDS FOR TREATING PNEUMONIA, SEPSIS, AND CORONAVIRUS INFECTION

(71) Applicants: HEIDELBERG IMMUNOTHERAPEUTICS GMBH, Heidelberg (DE); UNIVERSITÄT HEIDELBERG, Heidelberg (DE)

(72) Inventors: Torsten SCHALLER, Leimen (DE); Uta MERLE, Heidelberg (DE)

(21) Appl. No.: 17/917,801

(22) PCT Filed: Apr. 8, 2021
(86) PCT No.: PCT/EP2021/059201

§ 371 Date: Oct. 7, 2022

(30) Foreign Application Priority Data
Apr. 9, 2020 (EP) 20 16 9050.0
May 29, 2020 (EP) 20 17 7548.3

(51) Int. Cl. A61K 31/43; C07K 16/28; A61P 31/14

(52) U.S. Cl. CPC A61K 31/43; A61P 31/14; C07K 16/2866

(57) ABSTRACT

The present invention relates to an immune modulator comprising (i) a compound of formula I or a pharmaceutically acceptable salt or solvate thereof:

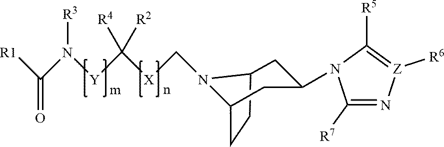

wherein X and Y are independently selected from —CH₂— and —O—, preferably are —CH₂—; Z is N or C, preferably is N; m is an integer of from 0 to 3, preferably is 0; n is an integer of from 1 to 3, preferably is 1; R¹ is, optionally substituted, C₅-C₆ cycloalkyl or aryl: R² is, optionally substituted, aryl or heteroaryl; R³ and R⁴ are —H, or R³ and R⁴ together form a —CH₂—CH₂— bridge; R⁵ and R⁷ are independently selected from alkyl, preferably methyl and isopropyl; and in case Z is N, R⁶ is absent, or, in case Z is C, R⁶ together with R⁵ forms a —CH═CH—CH═CH— bridge; and/or (ii) a C—C chemokine receptor type 5 (CCR5) inhibitor, for use in treating and/or preventing pneumonia, sepsis, and/or coronavirus infection in a subject, and to methods, uses, kits an devices related thereto.

The present invention relates to an immune modulator comprising (i) a compound of formula I or a pharmaceutically acceptable salt or solvate thereof:

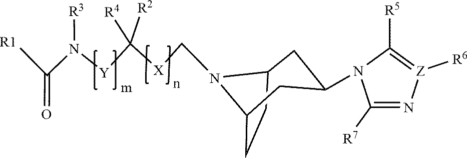

wherein X and Y are independently selected from —CH₂— and —O—, preferably are —CH₂—; Z is N or C, preferably is N; m is an integer of from 0 to 3, preferably is 0; n is an integer of from 1 to 3, preferably is 1; R¹ is, optionally substituted, C₅-C₆ cycloalkyl or aryl; R² is, optionally substituted, aryl or heteroaryl; R³ and R⁴ are —H, or R³ and R⁴ together form a —CH₂—CH₂— bridge; R⁵ and R⁷ are independently selected from alkyl, preferably methyl and isopropyl; and in case Z is N, R⁶ is absent, or, in case Z is C, R⁶ together with R⁵ forms a —CH═CH—CH═CH— bridge; and/or (ii) a C-C chemokine receptor type 5 (CCR5) inhibitor, for use in treating and/or preventing pneumonia, sepsis, and/or coronavirus infection in a subject, and to methods, uses, kits and devices related thereto.

In December 2019, the novel severe acute respiratory coronavirus 2 (SARS-CoV-2) was discovered to infect humans and has since lead to a pandemic of the associated coronavirus disease 2019 (COVID-19).

SARS-CoV-2 is a beta-coronavirus strain that causes unspecific and diverse disease courses, ranging from non-symptomatic infections to severe pneumonia, acute respiratory distress syndrome, and death. A limited number of laboratory metrics have been shown to correlate with disease course and are considered to potentially have prognostic value (Zhou F, Yu T, Du R, et al., Lancet 2020;395:1054-62). Physicians and scientists world-wide are working on therapeutic strategies, vaccine development, and prognostic markers. There are not yet any treatments proven effective, merely a very limited number of substances with emergency use authorizations or approval in some countries, very few promising experimental treatment approaches (Sanders JM, Monogue ML, Jodlowski TZ, Cutrell JB. Pharmacologic Treatments for Coronavirus Disease 2019 (COVID-19): A Review. JAMA 2020), very limited data on potential positive or negative effects on disease course of medication given for underlying conditions (Vaduganathan M, Vardeny O, Michel T, McMurray JJV, Pfeffer MA, Solomon SD., N Engl J Med 2020;382:1653-9), and no vaccines have been successfully tested in the short period of time between outbreak and today.

The Coronaviridae typically cause mild respiratory diseases, but infections with βcoronaviruses such as SARS-CoV-1, MERS, and SARS-CoV-2 can cause acute respiratory diseases and high mortalities, especially in individuals with underlying health conditions. In the last 20 years, Coronaviridae have emerged in three severe outbreaks, 2002/2003 with SARS-CoV-1, 2012 with MERS, and 2019/2020 with SARS-CoV-2. Multiple interventional clinical trials have been initiated in the search for effective pharmacological treatments against coronavirus disease 2019 (COVID-19) (Kupferschmidt & Cohen (2020), Science 367:1412, doi: 10.1126/science.367.6485.1412). Selection of single drug or combination treatments for COVID-19 trials has been based on previously described activities against SARS-CoV-1, Ebola, HIV and Malaria. Bioinformatics analyses have also proposed additional potential drugs based on the predicted interactome between viral proteins and host-cell pathways (Gordon et al. (2020), doi.org/10.1101/2020.03.22.002386v1). A frequent complication associated with coronavirus infection, in particular in COVID-19, is a severe pneumonia, which leads to sepsis in a high number of cases, both pneumonia and sepsis being the major causes of death in COVID-19.

C-C chemokine receptor type 5, also referred to as CCR5 or CD195, is a receptor on white blood cells with a still largely undefined role in the immune response. Notably, CCR5-positive inflammatory monocytes were found to be crucial for control of sepsis (Castanheira et al. (2019), Shock 52(5):e100). CCR5 became of interest as a pharmaceutical target when it was found that it is the co-receptor for certain strains of human immunodeficiency virus (HIV) type 1 and 2, and that its inhibition can prevent infection by these strains. As a consequence, CCR5 inhibitors such as Maraviroc, Vicriviroc, and Aplaviroc were developed.

A recent publication on compassionate use of remdesivir, a nucleotide analogue prodrug that inhibits viral RNA polymerases (Grein J, Ohmagari N, Shin D, et al., N Engl J Med 2020), indicates potential clinical benefits for patients with severe COVID-19. The antimalarial drugs chloroquine and hydroxychloroquine were reported to show benefits (Touret F, de Lamballerie X., Antiviral Res 2020;177:104762; Colson P, Rolain JM, Raoult D., Int J Antimicrob Agents 2020;55:105923; Gao J, Tian Z, Yang X., Biosci Trends 2020;14:72-3), however this view has been challenged by recent data and remains to be analyzed in randomized controlled clinical trials.

Recently, a phase 2, double blind, placebo controlled study on leronlimab, a humanized IgG4 monoclonal antibody that blocks the chemokine receptor CCR5 on immune cells, was initiated for treatment of COVID-19 patients with mild-to-moderate symptoms of respiratory illness (ClinicalTrials.gov Identifier: NCT04347239). On Apr. 24, 2020, this CCR5 inhibitor was also reported in compassionate use for six severely ill COVID-19 patients (Akalin E, Azzi Y, Bartash R, et al., N Engl J Med 2020). In this report, high levels of interleukin 6 (IL-6) in 5 of 6 patients were found to decrease substantially three days after administration of leronlimab. CCR5 blockade is known to decrease peripheral T-cell activation (Moy RH, Huffman AP, Richman LP, et al., Blood 2017;129:906-16).

Apart from leronlimab, CCR5 inhibition can be achieved with maraviroc, a CCR5 antagonist approved for the treatment of certain human immunodeficiency virus (HIV) infections. Maraviroc is also employed in oncology for immunomodulation via macrophage repolarization (Halama N, Zoernig I, Berthel A, et al., Cancer Cell 2016;29:587-601) (ClinicalTrials.gov Identifier: NCT01736813), and as an immunosuppressant in transplanted patients, preventing graft versus host disease (Reshef R, Luger SM, Hexner EO, et al., N Engl J Med 2012;367:135-45) (ClinicalTrials.gov Identifier: NCT00948753). These trials independently showed the safe use of maraviroc in different clinical settings and reported lack of significant drug-related toxicity.

There is, nonetheless, a need in the art for improved means and methods for treating and/or preventing coronavirus infection, pneumonia, and sepsis, avoiding the drawbacks of the prior art. This problem is solved by the means and methods disclosed herein with the features of the independent claims. Advantageous embodiments which might be realized in an isolated fashion or in any arbitrary combinations are listed in the dependent claims.

In accordance, the present invention relates to an immune modulator comprising
(i) a compound of formula I or a pharmaceutically acceptable salt or solvate thereof:

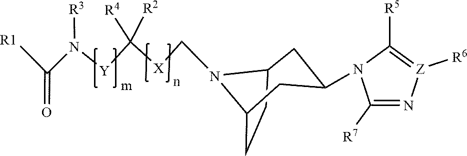

wherein
   X and Y are independently selected from —CH₂— and —O—, preferably are —CH₂—;
   Z is N or C, preferably is N;
   m is an integer of from 0 to 3, preferably is 0;
   n is an integer of from 1 to 3, preferably is 1;
   R¹ is, optionally substituted, C₅-C₆ cycloalkyl or aryl;
   R² is, optionally substituted, aryl or heteroaryl;
   R³ and R⁴ are —H, or R³ and R⁴ together form a —CH₂—CH₂— bridge;
   R⁵ and R⁷ are independently selected from alkyl, preferably methyl and isopropyl; and
   in case Z is N, R⁶ is absent, or,
   in case Z is C, R⁶ together with R⁵ forms a —CH═CH—CH═CH— bridge; and/or
(ii) a C-C chemokine receptor type 5 (CCR5) inhibitor,
   for use in treating and/or preventing pneumonia, sepsis, and/or coronavirus infection in a subject.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ± 20%, more preferably ± 10%, most preferably ± 5%. Further, the term “essentially” indicates that deviations having influence on the indicated result or use are absent, i.e. potential deviations do not cause the indicated result to deviate by more than ± 20%, more preferably ± 10%, most preferably ± 5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “%identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a %identity value of at least 80%, preferably at least 90%, more preferably at least 98%, most preferably at least 99%. As will be understood, the term essentially identical includes 100% identity. The aforesaid applies to the term “essentially complementary” mutatis mutandis. The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the immune modulators specified, in particular the CCR5 inhibitor polypeptides and CCR5 inhibitor polynucleotides, may be comprised in larger structures, e.g. may be covalently or non-covalently linked to carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C— or N— terminally to the fusion polypeptide of the present invention. Said stretch of amino acids preferably allows for detection of the fusion polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-Histag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and/or linker sequences.

As used herein, the term “immune modulator” relates to a compound of formula I or a pharmaceutically acceptable salt or solvate thereof and/or a C-C chemokine receptor type 5 (CCR5) inhibitor, both as specified elsewhere herein. The immune modulator has the biological activity of modulating the immune response of a subject, preferably of decreasing IL-6 and/or C-reactive protein (CRP) in the blood of a subject. Preferably, the immune modulator further has the activity of inhibiting CCR5 as specified herein below. The immune modulator may have any structure deemed appropriate by the skilled person, more preferably has a structure as specified herein.

Preferably, the immune modulator is a compound of formula I or a pharmaceutically acceptable salt or solvate thereof:

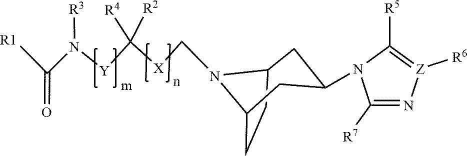

wherein
X and Y are independently selected from —CH₂— and —O—, preferably are —CH₂—;
Z is N or C, preferably is N;
m is an integer of from 0 to 3, preferably is 0;
n is an integer of from 1 to 3, preferably is 1;
R¹ is, optionally substituted, C₅-C₆ cycloalkyl or aryl;
R² is, optionally substituted, aryl or heteroaryl;
R³ and R⁴ are —H, or R³ and R⁴ together form a —CH₂—CH₂— bridge;
R⁵ and R⁷ are independently selected from alkyl, preferably methyl and isopropyl; and
in case Z is N, R⁶ is absent, or,
in case Z is C, R⁶ together with R⁵ forms a —CH═CH—CH═CH— bridge.

The term “side chain” is understood by the skilled person and relates to an atom or chemical group attached covalently to the core part of a chemical compound as described herein, said core part also being referred to as “main chain” or “backbone”. Preferably, the side chain is an organic side chain as described herein below. The term “substituted” side chain relates to a side chain substituted at one or more positions, preferably, at 1, 2, or 3 positions, wherein substituents may be attached at any available atom to produce a stable chemical compound. It is understood by the skilled person that the term “optionally substituted” side chain relates to an unsubstituted or to a substituted side chain.

The term “organic side chain”, as used herein, relates to any, optionally substituted, side chain comprising at least one carbon atom. Preferably, the organic side chain is an, optionally substituted, alkyl, alkenyl, alkinyl, aryl, aralkyl, cycloalkyl, heterocycloalkyl, or heteroaryl side chain. Preferably, a substituted organic side chain is an organic side chain substituted with at least one substituent independently selected from —COO^{—}, ═O, —OH, —CN, halogen, —NH₂, -NH(alkyl), —N(alkyl)₂, —N(alkyl)₃⁺, —NH(aryl), N(aryl)₂, —NO₂, —O(alkyl), —O—(CH₂)ₙ—OH, —O—(CH₂)n—O(alkyl), —O(aralkyl), —O(aryl), —OPO₃²⁻, —PO₃²⁻, —OSO₃⁻ and —SO₃^{—}. Preferably, the alkyl, aryl, and aralkyl groups of the substituents are not further substituted by groups comprising alkyl, alkenyl, alkinyl, aryl, aralkyl, heterocycloalkyl, or heteroaryl groups. More preferably, the alkyl, aryl, and aralkyl groups of the substituents are not further substituted.

The term “alkyl”, as used herein, relates to a straight or branched chain, saturated hydrocarbon group, linked to the main chain by a covalent bond to at least one of its at least one carbon atoms. Preferred alkyl groups are straight chain alkyls, e.g., preferably, methyl, ethyl, propyl, butyl, pentyl, hexyl, heptyl, octyl, nonyl, decyl, undecyl, dodecyl, or branched chain alkyl groups, e.g., preferably, —CH(CH₃)₂, —CH(CH₂CH₃)₂, —C(CH₃)₃, —C(CH₂CH₃)₃, —CH(CH₃)(CH₂CH₃), —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH3)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, or —CH(CH₃)CH(CH₃)CH(CH₃)₂. Accordingly, alkyl groups include primary alkyl groups, secondary alkyl groups, and tertiary alkyl groups. Preferably, alkyl is methyl and/or isopropyl. The term “cycloalkyl” relates to a circularly closed, hydrocarbon group, preferably with 3 to 12 carbon atoms. Preferred cycloalkyls are, optionally substituted, cyclopropyl, cyclobutyl, cyclopentyl, cyclohexyl, cycloheptyl, and cyclooctyl. More preferably, cycloalkyl is, optionally substituted cyclohexyl, more preferably is Fluoro or Difluoro-cyclohexyl, most preferably is 4,4-difluoro-cyclohexyl.

The term “alkenyl” side chain relates to a side chain comprising at least one C═C double bond and linked to the main chain by a covalent bond to at least one of its at least two carbon atoms. Accordingly, the term “alkinyl” side chain relates to a side chain comprising at least one C═C triple bond linked to the main chain by a covalent bond to at least one of its at least two carbon atoms.

The term “cycloalkenyl” relates to a circularly closed hydrocarbon group, preferably with 5 to 12 carbon atoms, comprising at least one C═C double bond and linked to the main chain by a covalent bond to at least one of its at least two carbon atoms. The term “cycloalkinyl” relates to a circularly closed hydrocarbon group, preferably with 8 to 12 carbon atoms, comprising at least one C═C triple bond and linked to the main chain by a covalent bond to at least one of its at least two carbon atoms.

As used herein, the term “alkoxy” side chain relates to an —O—alkyl side chain, preferably having the indicated number of carbon atoms. Preferably, the alkoxy side chain is —O—methyl, —O—ethyl, —O—propyl, —O—isopropyl, —O—butyl, —O—sec-butyl, —O—tert-butyl, —O—pentyl, —O—isopentyl, —O—neopentyl, —O—hexyl, —O— isohexyl, or —O—neohexyl.

The term “aryl”, as used herein, relates to an aromatic ring or ring system having 6 to 14 carbon atoms, preferably comprising one, two, or three aromatic rings. Preferred aryl side chains are, optionally substituted, phenyl, naphthyl, anthracenyl, and phenanthrenyl. More preferably, aryl is phenyl or 2-fluorophenyl. The term “ring”, in the context of the chemical compounds of the present invention, is understood by the skilled person; accordingly, the term “ring system” relates to a chemical structure comprising at least two rings sharing at least one covalent bond. Thus, preferably, “aryl” also includes aromatic ring systems fused with a cycloalkyl and/or a heterocycloalkyl ring.

As used herein, the tem “aralkyl” relates to an alkyl side chain, wherein at least one hydrogen is replaced by an aryl side chain. Preferably, aralkyl is benzyl or phenethyl.

The term “heterocycloalkyl”, as used herein, relates to a saturated or partially unsaturated ring or ring system having 5 to 14 ring atoms, preferably 5 to 7 ring atoms, wherein at least one ring atom is a heteroatom selected from the group consisting of N, O, and S, said ring or ring system being linked to the main chain by a covalent bond to a C or N atom of said ring or ring system. Preferably, heterocycloalkyl is azepinyl, dihydrofuryl, dihydropyranyl, imidazolidinyl, imidazolinyl, isothiazolidinyl, isoxazolidinyl, morpholinyl, oxazolidinyl, piperazinyl, piperidinyl, pyrazolidinyl, pyrrolidinyl, tetrahydrofuryl, tetrahydropyranyl, thiadiazolylidinyl, thiazolidinyl, or thiomorpholinyl.

As used herein, the term “heteroaryl” relates to an aromatic ring or ring system having 5 to 14 ring atoms, preferably 5 to 7 ring atoms, wherein at least one ring atom is a heteroatom selected from the group consisting of N, O, and S, said ring or ring system being linked to the main chain by a covalent bond to a C or N atom of said ring or ring system. Preferably, up to 4, more preferably up to 3, most preferably up to 2 ring atoms per ring are heteroatoms independently selected from the group of heteroatoms consisting of N, O, and S. Preferably, heteroaryl is pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, or indolyl.

Preferably, in formula I, X is —CH₂—. Preferably, in formula I, n is 1. Preferably, in formula I, Z is N. Preferably, in formula I, m is 0, i.e. Y is absent. Preferably, in formula I, R3 and R4 are —H. Preferably, in formula I, R¹ is optionally substituted cycloalkyl, preferably is 4,4-difluoro-cyclohexanyl. Preferably, in formula I, R² is aryl, preferably phenyl. Preferably, in formula I, R⁵ is methyl and R⁷ is isopropyl. Preferably, in formula I, X is —CH₂— and n is 1. Preferably, in formula I, X is —CH₂— and Z is N. Preferably, in formula I, X is —CH₂— and m is 0. Preferably, in formula I, X is —CH₂— and R³ and R⁴ are —H. Preferably, in formula I, X is —CH₂— and R¹ is optionally substituted cycloalkyl, preferably is 4,4-difluoro-cyclohexanyl. Preferably, in formula I, X is —CH₂— and R² is aryl, preferably phenyl. Preferably, in formula I, X is —CH₂—, R⁵ is methyl and R⁷ is isopropyl. Preferably, in formula I, X is —CH₂—, n is 1, and Z is N. Preferably, in formula I, X is —CH₂—, n is 1, and m is 0. Preferably, in formula I, X is —CH₂—, n is 1, and R³ and R⁴ are —H. Preferably, in formula I, X is —CH₂—, n is 1, and R¹ is optionally substituted cycloalkyl, preferably is 4,4-difluoro-cyclohexanyl. Preferably, in formula I, X is —CH₂—, n is 1, and R² is aryl, preferably phenyl. Preferably, in formula I, X is —CH₂—, n is 1, R⁵ is methyl and R⁷ is isopropyl. Preferably, in formula I, X is —CH₂—, n is 1, Z is N, and m is 0. Preferably, in formula I, X is —CH₂—, n is 1, Z is N, and R³ and R⁴ are —H. Preferably, in formula I, X is —CH₂—, n is 1, Z is N, and R¹ is optionally substituted cycloalkyl, preferably is 4,4-difluoro-cyclohexanyl. Preferably, in formula I, X is —CH₂—, n is 1, Z is N, and R² is aryl, preferably phenyl. Preferably, in formula I, X is —CH₂—, n is 1, Z is N, and R⁵ is methyl and R⁷ is isopropyl. Preferably, in formula I, X is —CH₂—, n is 1, Z is N, m is 0, and R³ and R⁴ are —H. Preferably, in formula I, X is —CH₂—, n is 1, Z is N, m is 0, and R¹ is optionally substituted cycloalkyl, preferably is 4,4-difluoro-cyclohexanyl. Preferably, in formula I, X is —CH₂—, n is 1, Z is N, m is 0, and R² is aryl, preferably phenyl. Preferably, in formula I, X is —CH₂—, n is 1, Z is N, m is 0, R⁵ is methyl and R⁷ is isopropyl. Preferably, in formula I, X is —CH₂—, n is 1, Z is N, m is 0, R³ and R⁴ are —H, and R¹ is optionally substituted cycloalkyl, preferably is 4,4-difluoro-cyclohexanyl. Preferably, in formula I, X is —CH₂—, n is 1, Z is N, m is 0, R³ and R⁴ are —H, and R² is aryl, preferably phenyl. Preferably, in formula I, X is —CH₂—, n is 1, Z is N, m is 0, R³ and R⁴ are —H, R⁵ is methyl and R⁷ is isopropyl. Preferably, in formula I, X is —CH₂—, n is 1, Z is N, m is 0, R³ and R⁴ are —H, R¹ is 4,4-difluoro-cyclohexanyl, and R² is aryl, preferably phenyl. Preferably, in formula I, X is —CH₂—, n is 1, Z is N, m is 0, R³ and R⁴ are —H, R¹ is 4,4-difluoro-cyclohexanyl, R⁵ is methyl and R⁷ is isopropyl. Preferably, in formula I, X is —CH₂—, n is 1, Z is N, m is 0, R³ and R⁴ are —H, R¹ is 4,4-difluoro-cyclohexanyl, and R² is phenyl, R5 is methyl and R⁷ is isopropyl. More preferably, the compound is Maraviroc (4,4-difluoro—N—[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide, CAS NO: 376348-65-1).

Preferably, m is 0, n is 1, X is —CH₂—, Z is N, R¹ is 4,4-difluoro-cyclohexanyl, R² is Thiophen-3-yl, R³ and R⁴ are —H, R⁵ is methyl, and/or R⁷ is isopropyl. More preferably, the compound is CCR5 antagonist 34 (4,4-difluoro—N—[(1S)-3-[(1S,5R)-3-(3-methyl-5-propan-2-yl-1,2,4-triazol-4-yl)-8-azabicyclo[3.2.1]octan-8-yl]-1-thiophen-3-ylpropyl]cyclohexane-1-carboxamide, PubChem CID 91820689).

Preferably, m is 2, n is 1, X is —CH₂—, Y is —CH₂—, Z is C, R¹ is N-tert-butyl-4-chloro-2-fluorobenzene-1-sulfonamide-5-yl, R² is 2-flourophenyl, R³ and R⁴ together form a —CH₂—CH₂—bridge, R⁶ together with R⁵ forms a —CH═CH—CH═CH— bridge, and/or R⁷ is methyl. More preferably, the compound is CCR5 antagonist 1 (CAS NO: 716354-86-8).

Preferably, the immune modulator is a compound of formula II or III:

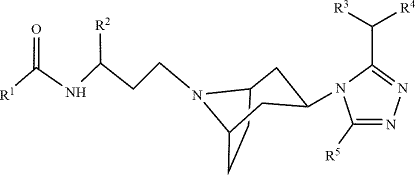

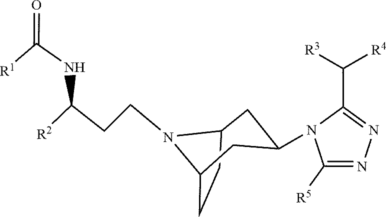

wherein R₁ is C₃₋₆ cycloalkyl, optionally substituted by one or more fluorine atoms, or C₁₋₄ alkyl optionally substituted by one or more fluorine atoms, or C₃₋₈ cycloalkylmethyl, optionally ring-substituted by one or more fluorine atoms; and R² is phenyl optionally substituted by one or more fluorine atoms: Preferred compounds according to formulas II and III and methods for their preparation are also described e.g. in U.S. Pat. 7,576,097 B2. more preferably, R1 is R1 as specified herein above, and/or R² is R² as specified herein above.

The terms “C-C chemokine receptor type 5” and its abbreviation “CCR5” are understood by the skilled person to relate to the member of the family of beta chemokine receptors known under this designation. Preferably, CCR5 is the human CCR5, having an amino acid sequence as shown in Genbank Acc No. NP_000570.1 or NP_001093638.1, or a polypeptide being at least 70%, more preferably at least 80%, still more preferably at least 90%, most preferably at least 95% identical to one of said sequences; preferably, said polypeptide having the aforesaid identity is a homolog of human CCR5, more preferably a mammalian homolog. Most preferably, CCR5 is the human CCR5.

Thus, the CCR5 comprises a polypeptide with at least 70% identity to the Genebank reference sequence NP_001093638.1.

Accordingly, the CCR5 comprises a polypeptide with at least 70% identity to SEQ ID NO:1 wherein the polypeptide has the activity of CCR5.

In a more preferred embodiment, the CCR5 comprises a polypeptide which is at least n % identical to the above sequence with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99.

As regards the determination of the sequence identity, the same applies as has been set forth above.

Assays for determining the activity of CCR5 are known in the art and are described in the following in the context of the inhibition of CCR5.

In accordance with the above, the term “CCR5 inhibitor”, as used herein, includes any and all compounds causing the activity of CCR5 in a cell, preferably of human CCR5 in a human cell, to decrease. Assays for determining CCR5 inhibition are known in the art, e.g., from Kim et al. (2016), Expert Opin Investig Drugs 25(12):1377. Preferably, the activity of a CCR5 inhibitor is determined in a competitive assay measuring displacement of radiolabeled RANTES (CCL5) from CCR5; more preferably, the activity of a CCR5 inhibitor is determined in a cell-based assay determining calcium mobilization after CCR5 activation. The effect of the CCR5 inhibitor may be indirect, e.g. by decreasing the amount of CCR5 in cells, or may be direct, i.e., preferably, by a direct interaction between CCR5 and the CCR inhibitor. Thus, preferably, the CCR5 inhibitor is a CCR5 antagonist.

Preferably, the CCR5 inhibitor is a high-molecular weight compound, preferably a biological macromolecule, more preferably a polynucleotide or a polypeptide. In case the CCR5 inhibitor is a polynucleotide, it may be a direct inhibitor, e.g. a polynucleotide aptamer binding to the receptor region of CCR5; the polynucleotide may also be an indirect inhibitor, causing the amount of CCR5 in a cell to decrease. The principles for providing a polynucleotide suitable for indirectly inhibiting a cellular receptor are known to the skilled person and include in particular provision of siRNAs, miRNA, shRNAs, and the like, decreasing expression of said receptor. Polypeptides and peptides inhibiting CCR5, preferably being direct CCR5 inhibitors, are also known to the skilled person, and include antibodies specifically recognizing the receptor region of CCR5, in particular Leronlimab (PRO 140), viral macrophage inflammatory protein-II (PubchemID: 486830, Genbank Acc No. Q98157.1), and CCL7 (e.g. human CCL7: Genbank Acc No. EAW80210.1).

More preferably, the CCR5 inhibitor is a low-molecular weight compound, preferably having a molecular mass of at most 1 kDa. Appropriate assays for identifying low molecular weight CCR5 inhibitors are known in the art and are referred to elsewhere herein. Preferably, the CCR5 inhibitor is selected from the list consisting of
Maraviroc (4,4-difluoro-N-[(1S)-3-[(1R,SS)-3-(3-methyl-5-propan-2-yl-1,2,4-triazol-4-yl)-8-azabicyclo[3.2.1]octan-8-yl]-1-phenylpropyl]cyclohexane-1-carboxamide, CAS NO: 376348-65-1),
Vicriviroc ((4,6-dimethylpyrimidin-5-yl)-[4-[(3S)-4-[(1R)-2-methoxy-1-[4-(trifluoromethyl)phenyl]ethyl]-3-methylpiperazin-1-yl]-4-methylpiperidin-1-yl]methanone, CAS NO: 306296-47-9),
Aplaviroc (4-[4-[[(3R)-1-butyl-3-[(R)-cyclohexyl-hydroxymethyl]-2,5-dioxo-1,4,9-triazaspiro[5.5]undecan-9-yl]methyl]phenoxy]benzoic acid, CAS NO: 461443-59-4), ancriviroc ([4-[4-[(Z)-C-(4-bromophenyl)-N-ethoxycarbonimidoyl]piperidin-1-yl]-4-methylpiperidin-1-yl]-(2,4-dimethyl-1-oxidopyridin-1-ium-3-yl)methanone, CAS NO: 370893-06-4),
Cenicriviroc ((SE)-8-[4-(2-butoxyethoxy)phenyl]-1-(2-methylpropyl)-N-[4-[(S)-(3-propylimidazol-4-yl)methylsulfinyl]phenyl]-3,4-dihydro-2H-1-benzazocine-5-carboxamide, CAS NO: 497223-25-3),
TAK-779 (dimethyl-[[4-[[3-(4-methylphenyl)8,9-dihydro-7H-benzo[7]annulene-6-carbonyl]amino]phenyl]methyl]-(oxan-4-yl)azanium, CAS NO: 263765-56-6),
E913 (1-butyl-3-(cyclohexylmethyl)-9-(2,3-dihydro-1,4-benzodioxin-7-ylmethyl)-1,4,9-triazaspiro[5.5]undecane-2,5-dione, CAS NO: 342394-93-8),
TAK-220 (1-acetyl-N-[3-[4-[(4-carbamoylphenyl)methyl]piperidin-1-yl]propyl]-N-(3-chloro-4-methylphenyl)piperidine-4-carboxamide, CAS NO:333994-00-6 ),
AZD5672 (N-[1-[(3R)-3-(3,5-difluorophenyl)-3-(1-methylsulfonylpiperidin-4-yl)propyl]piperidin-4-yl]-N-ethyl-2-(4-methylsulfonylphenyl)acetamide, PubChem CID: 11614352),
BMS-681 ((3S)-1-[(1S,2R,4R)-4-[methyl(propan-2-yl)amino]-2-propylcyclohexyl]-3-{ [6-(trifluoromethyl)quinazolin-4-yl]amino}pyrrolidin-2-one, PubChem CID: 68764898),
CCR5 antagonist 34 (4,4-difluoro-N-[(1S)-3-[(1S,SR)-3-(3-methyl-5-propan-2-yl-1,2,4-triazol-4-yl)-8-azabicyclo[3.2.1]octan-8-yl]-1-thiophen-3-ylpropyl]cyclohexane-1-carboxamide, PubChem CID:91820689),
GSK2239633A (N-[[3-[[3-[(5-chlorothiophen-2-yl)sulfonylamino]-4-methoxyindazol-1-yl]methyl]phenyl]methyl]-2-hydroxy-2-methylpropanamide, CAS NO: 1240516-71-5), and
INCB009471 ((4,6-dimethylpyrimidin-5-yl)-[4-[(3S,4R)-4-[(2R)-2-ethoxy-5-(trifluoromethyl)indan-1-yl]-3-methyl-piperazin-1-yl]-4-methyl-1-piperidyl]methanone, CAS NO: 925701-76-4).

More preferably, the CCR5 inhibitor is selected from the list consisting of Maraviroc, Vicriviroc, and Aplaviroc, still more preferably, is Maraviroc.

The term “treating”, as used herein, refers to ameliorating or curing a disease or at least one symptom associated therewith. Thus, if there is amelioration or cure of the disease or at least a symptom associated therewith, the treatment shall be deemed to be effective. It will be understood that treating may not be effective in all subjects. However, according to the present invention it is envisaged that treatment will preferably be effective in at least a statistically significant portion of subjects to be treated. Whether a portion is statistically significant can be determined without further ado by the person skilled in the art using various well known statistic evaluation tools, e.g., determination of confidence intervals, p-value determination, Student’s t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the finding of coronavirus infection will be correct for at least 60%, at least 70%, at least 80%, or at least 90% of the subjects of a given cohort or population.

The term “preventing” as used herein refers to avoiding the onset of the disease or at least one symptom associated therewith or to prevent the worsening of the disease or the said at least one symptom. The prevention as referred to herein can be typically achieved shortly after the compound is administered. If the administration stopped, however, the prevention may not persist for an unlimited time but may remain present for a certain preventive time window after application of the drug. Typically, a preventive time window in accordance with the present invention may be at least 1 day, at least 2 days, at least 3 days, at least 4 days, at least 5 days, at least 7 days, or at least two or four weeks. However, the preventive time window may also depend on the dosage as well as the mode of administration, the kind of formulation, and/or the number of administrations. For example, if a high dosage is applied, usually longer preventive time windows can be achieved. The same holds true if repeated doses are administered. It will be understood that prevention might not be effective in all subjects. However, according to the present invention it is envisaged that prevention preferably will be effective in a statistically significant portion of subjects. Whether a portion is statistically significant can be determined without further ado by the person skilled in the art using various well known statistic evaluation tools as discussed above. In view of the above, prevention, preferably, is vaccination against coronavirus infection.

Preferably, treating and/or preventing, more preferably treating, as referred to herein comprises administration of further treatments to a subject. Such treatment optionally administered in addition to administration of a compound referred to herein preferably depends on severity of the disease and its symptoms and is preferably selected appropriately by the medical practitioner. Thus, such additional treatment may comprise assistance with breathing, in particular administration of increased oxygen partial pressure, intubation, artificial respiration, treatment of sepsis, preferably as specified herein below, and the like. Further, treating and/or preventing may comprise administration of further pharmaceuticals, in particular compounds known or suspected to be suitable in the relevant treatment; thus, the compounds referred to herein may be combined with compounds known or suspected to treat coronavirus infection, such as Favipiravir (Avigan®), Camostat, Cyclosporine, Emetine, Loratadine, Monensin, Nafamostat, Pevonedistat, Carboxyamidotriazole, Thioguanine, and/or Remdesivir.

Further, treating and/or preventing may comprise administration of yet further pharmaceuticals, in particular compounds known or suspected to be suitable in the relevant treatment; thus, the compounds referred to herein may also be combined with compounds known or suspected to treat coronavirus infection, such as Losartan, acetylsalicylic acid (Aspirin), Umifenovir (Arbidol), Ribavirin, Ritonavir, and/or Lopinavir.

Also, the compounds referred to herein may be combined with compounds known or suspected to treat pneumonia, in particular antibiotics such as erythromycin, and/or antiviral compounds such as Favipiravir. Further the compounds may be combined with piperacillin/tazobactam, caspofungin, and/or hydroxychloroquine. Also, the compounds referred to herein may be combined with compounds known or suspected to treat sepsis, such as antibiotics, e.g. erythromycin, antiviral compounds such as Favipiravir, intravenous fluids, blood products, in particular comprising erythrocytes, vasopressors, e.g. Norepinephrine or Dobutamine, and/or steroids. As the skilled person understands, in the treatment and/or prevention referred to herein, also a combination of two or more CCR5 inhibitors, preferably as specified herein, may be used.

In further preferred embodiments, the treating and/or preventing comprises the administration of an (additional) compound against bacterial, fungal, and/or viral superinfections in the form of a combination therapy with the above-defined immune modulator, preferably a CCR5 inhibitor of the present invention.

Corresponding suitable to treat or prevent bacterial, fungal, and/or viral superinfections are well-known to the person skilled in the art and a compound against bacterial, fungal, and/or viral superinfections in accordance with the present invention is not limited to specific compounds against bacterial, fungal, and/or viral superinfections. Rather, the skilled person is in a position to select a suitable compound against bacterial, fungal, and/or viral superinfections as long as it is capable of treating and/or preventing bacterial, fungal, and/or viral superinfections.

Preferably, the compound against bacterial, fungal, and/or viral superinfections is piperacillin/tazobactam or caspofungin.

In further preferred embodiments, the treating and/or preventing comprises the administration (in the form of a combination therapy with the above-defined immune modulator, preferably a CCR5 inhibitor of the present invention) of a compound selected from the group consisting of meropenem, azithromycin, vancomycin, co-trimoxazole, ampicillin/sulbactam, ceftriaxone, flucloxacilin, moxifloxacin, aciclovir, ganciclovir, and voriconazole.

In yet further preferred embodiments, the treating and/or preventing comprises the administration (in the form of a combination therapy with the above-defined immune modulator, preferably a CCR5 inhibitor of the present invention) of a compound selected from the group consisting of hydroxychloroquine (HCQ), prednisolone, intravenous immunoglobulin (IVIG), tocilizumab, and plasmapheresis.

Preferably, such a combination therapy exerts synergistic effects on the treatment in accordance with the present invention.

As regards the preferred embodiments of such a combination therapy, the same applies, mutatis mutandis, as has been set forth above in the context of the immune modulator (preferably a CCR5 inhibitor of the present invention) for use in treating and/or preventing pneumonia, sepsis and/or coronavirus infection in a subject as defined above.

The term “combination” as used herein relates to a combination of an immune modulator (preferably a CCR5 inhibitor of the present invention) for use in pneumonia, sepsis and/or coronavirus infection in a subject as described herein above and an additional compound (i.e., a compound against bacterial, fungal, and/or viral superinfections) described herein above.

In a preferred embodiment, a simultaneous application is envisaged. Yet, the combination also encompasses a subsequent application of the two components. Thus, one of these components may be administered before, simultaneously with or after the other one of the combination, or vice versa.

Accordingly, “in combination” as used herein does not restrict the timing between the administration of the immune modulator (preferably a CCR5 inhibitor) of the present invention as outlined above and the above additional compound (i.e., a compound against bacterial, fungal, and/or viral superinfections). Thus, when the two components are not administered simultaneously with/concurrently, the administrations may be separated by 1 minute, 5 minutes, 15 minutes, 30 minutes, 45 minutes, 1 hour, 2 hours, 4 hours, 6 hours, 12 hours, 24 hours, 48 hours or 72 hours or by any suitable time differential readily determined by one of skill in art and/or described herein. In a preferred embodiment, when the two components are not administered simultaneously with/concurrently, the administrations may be separated by 1 hour, 2 hours, 4 hours, 6 hours, 12 hours, 24 hours, 48 hours or 72 hours or by any suitable time differential readily determined by one of skill in art and/or described herein.

Preferably, treating and/or preventing as referred to herein comprises modulating an immune response of a subject, in particular in case of treatment and/or prevention of sepsis; thus, treating and/or preventing preferably comprises reducing the severity of the hyper-inflammatory response in sepsis, more preferably by inhibition of T-cell migration to inflammation sites and/or by reducing antigen presentation by antigen presenting cells (APC).

In another preferred embodiment, treating and/or preventing preferably comprises reducing the severity of the hyper-inflammatory response in sepsis, more preferably by inhibition of leukocyte migration to inflammation sites and/or by reducing antigen presentation by antigen presenting cells (APC).

Also preferably, treating and/or preventing comprises reducing severity of the hypo-inflammatory phase of sepsis, in particular comprises increasing monocyte counts, preferably macrophage counts, at inflammation sites. Also preferably, treating and/or preventing comprises inhibition of tissue destruction, preferably lung tissue destruction, and/or inhibition of cell lysis. Preferably, treating and/or preventing comprises inhibition of coronavirus replication, preferably by improving the cellular immune response against infected cells. Also, preferably, treating and/or preventing comprises inhibition of cell lysis caused by the coronavirus.

As is understood by the skilled person, the term “inhibition” includes partial inhibition. Thus, in the case of a quantifiable event, inhibition preferably is a reduction of said event by at least 25%, more preferably at least 50%, still more preferably at least 75%, most preferably at least 85%. As will be understood by the skilled person, inhibition may also include complete inhibition, i.e. prevention of an event or process from occurring. Exemplary methods for determining inhibition by the compounds specified herein are provided elsewhere herein.

The term “coronavirus” is understood by the skilled person to relate to a group of enveloped viruses from the order Nidovirales, having a positive-sense single-stranded RNA genome with a size of approx. 25 to 35 kilobases. Preferably, the coronavirus is a beta-coronavirus, more preferably a severe acute respiratory syndrome coronavirus (SARS-CoV-2, SARS-CoV-1, or Middle East respiratory syndrome coronavirus (MERS-CoV). Preferably the coronavirus is SARS-CoV-2. The terms “SARS-CoV-2” and “severe acute respiratory syndrome coronavirus 2” are understood by the skilled person. Preferably, the terms relate to the virus identified in Genbank entry NCBI:txid2697049.

In a more preferred embodiment, the term “SARS-CoV-2” comprises viruses with at least 70% identity in the amino acid sequences of their expressed open reading frames to the Genebank reference sequence NC_045512.2.

More specifically, the term “SARS-CoV-2” comprises viruses with at least 70% identity in the complete genome sequence of the Genebank reference sequence NC_045512.2 (SEQ ID NO:2). In a more preferred embodiment, the term “SARS-CoV-2” comprises a genomic sequence of SEQ ID NO:2 which is at least n % identical to the above sequence with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99.

As regards the determination of the sequence identity, the same applies as has been set forth above.

Symptoms and diseases caused by coronavirus infection and in particular SARS-CoV-2 infection are known to the skilled person. Also preferably the coronavirus is SARS-CoV-1 or Middle East respiratory syndrome coronavirus (MERS-CoV, NCBI:txid1335626).

In a more preferred embodiment, the term “MERS-CoV” comprises viruses with at least 70% identity in the amino acid sequences of their expressed open reading frames to the Genebank reference sequence NC_019843.3.

More specifically, the term “MERS-CoV” comprises viruses with at least 70% identity in the complete genome sequence of the Genebank reference sequence NC_019843.3 (SEQ ID NO:3). In a more preferred embodiment, the term “MERS-CoV” comprises a genomic sequence of SEQ ID NO:3 which is at least n % identical to the above sequence with n being an integer between 10 and 100, preferably 10, 15, 20, 25, 30, 35, 40, 45, 50, 55, 60, 65, 70, 75, 80, 85, 90, 91, 92, 93, 94, 95, 96, 97, 98 or 99.

As regards the determination of the sequence identity, the same applies as has been set forth above.

In accordance, the term “coronavirus infection” relates to infection of a subject with a coronavirus as specified elsewhere herein. Preferably, the coronavirus infection is or is associated with pneumonia, and/or sepsis, i.e., preferably, is coronavirus-associated pneumonia and/or coronavirus-associated sepsis.

The term “human immunodeficiency virus” (HIV) is understood by the skilled person to relate to the group of retroviruses known under this designation, e.g. under NCBI:txid12721 (HIV), NCBI:txid1167 (HIV-1), NCBI:txid11709 (HIV-2), or NCBI:txid35274 (HIV-3). Preferably, the HIV is a strain using the CCR5 receptor as entry mediator.

The term “pneumonia” is understood by the skilled person and typical causes, symptoms, and treatments can be derived from medical textbooks. Preferably, the pneumonia is a viral, bacterial, or yeast pneumonia, more preferably a viral pneumonia. Preferably, pneumonia is a coronavirus-associated pneumonia, preferably a SARS-CoV-2 associated pneumonia. Also preferably, the pneumonia is a HIV-associated pneumonia, preferably a Pneumocystis pneumonia. Also preferably, the pneumonia is a pneumonia caused by or associated with infection by Streptococcus pneumoniae, Haemophilus influenzae, Chlamydophila pneumonia, Chlamydia psittaci, Mycoplasma pneumonia, Staphylococcus aureus, in particular methicillin-resistant Staphylococcus aureus, Moraxella catarrhalis, Legionella pneumophila, and/or Gram-negative bacilli, such as Pseudomonas aeruginosa and Coxiella burnetti. As the skilled person understands, pneumonia may be accompanied by secondary, often opportunistic superinfections. In accordance, the terms “viral pneumonia” and “virus associated pneumonia”, as used herein, relate to any pneumonia in which a pneumonia-causing virus is detectable, irrespective of whether it is the agent of primary infection, or not.

The term “sepsis” is understood by the skilled person and typical causes, symptoms, and treatments can be derived from medical textbooks. Preferably, the term refers to an inflammatory response affecting the entire organism, symptoms preferably including one or more of a significantly altered body temperature (low temperature or fever), rapid breathing, tachycardia, low blood pressure due to decreased peripheral vascular resistance, mental confusion and edema formation. Biochemical parameters such as coagulation dysfunction or metabolic acidosis are also typical signs of sepsis. Sepsis preferably is caused by severe infection by bacteria, viruses, parasites or fungi. Moreover, there are cofounding factors which influence the onset or outcome of sepsis, such as diabetes or cancer. Preferably, sepsis as referred to herein is characterized by the presence of two or more of the following symptoms in response to an infection: abnormal temperature (preferably, below 36° C. or above 38° C.), abnormal heart rate (preferably, above 90 beats/min), abnormal respiratory rate (preferably, above 20 breathings/min) or blood gas composition (preferably, CO2 less than 4.3 kPa), and abnormal white blood cell number (preferably, less than 4x10⁹/L or more than 12x10⁹/L or histological presence of band neutrophils). More preferably, the sepsis is caused by a viral, bacterial, or yeast infection in a subject, preferably by a viral, bacterial, or yeast pneumonia. Preferably, sepsis is a virus-associated sepsis. More preferably, the sepsis is a coronavirus-associated sepsis, still more preferably SARS-CoV-2 associated sepsis. Also more preferably, the sepsis is HIV-associated sepsis. As the skilled person understands, sepsis may be accompanied or caused by mixed-type or secondary infections. In accordance, the terms “viral sepsis” and “virus-associated sepsis”, as used herein, relate to any sepsis in which an agent referred to herein is detectable, irrespective of whether it is the agent of primary infection, or not. Preferably, sepsis includes sepsis-associated organ failure.

The term “organ failure”, as used herein, refers to any dysfunction of the organ which affects the physiologically expected function of an organ to such an extent that normal homeostasis can neither be maintained nor endogenously compensated. Organ failure may be acute or chronic, preferably is acute. Symptoms associated with organ failure depend on the affected organ usually become apparent by a pathological physiology in the subject which can be determined, e.g., by clinical or biochemical parameters. Symptoms of organ failure are also well known in the art and are described in medicinal text books. Preferably, organ failure as referred to herein is multi organ failure. Multi organ failure is characterized by the failure of two or more organs at the same time or sequentially within a short period of time. It can be often observed as a consequence of severe infections or inflammatory reactions such as sepsis.

Typical organs which fail during sepsis are lung, kidney, heart and/or the entire circulation system, the gastrointestinal system, including in particular the liver, as well as the nervous system. Preferably, the multi organ failure referred to herein is caused by autoreactive cytotoxic cells and, more preferably, is CD8 cytotoxic T-cell dependent multi-organ failure. Also preferably, organ failure as referred to herein is liver failure, in particular liver failure in septic subjects.

The term “subject” as used herein refers to any kind of animal encompassing a CCR5, e.g., mammals, birds, fish or reptiles. Typically, the animal, however, is a mammal such as a mammal used as a pet, including dogs, cats, horses, or rodents, laboratory animals, e.g., rats, mice or apes, or farming animals such as pigs, cows, goats, or sheep. More preferably, the mammal is a primate and, most preferably, a human. The subject according to the present invention shall preferably be known or suspected to suffer from coronavirus infection, from pneumonia, and/or sepsis. More preferably, the subject is known or suspected to suffer from coronavirus associated pneumonia and/or coronavirus associated sepsis. Also preferably, the subject according to the present invention is known or suspected to suffer from HIV infection, from pneumonia, and/or sepsis. More preferably, the subject is known or suspected to suffer from HIV associated pneumonia and/or HIV associated sepsis. Preferably, the subject is not homozygous for the CCR5Δ32 allele; thus, preferably, the subject carries at most one CCR5Δ32 allele. Preferably, the subject receives treatment with piperacillin/tazobactam, treatment with caspofungin, or treatment with hydroxychloroquine; more preferably receives treatment with piperacillin/tazobactam and caspofungin; most preferably receives treatment with piperacillin/tazobactam, caspofungin and hydroxychloroquine.

Preferably, the CCR5 inhibitor is comprised in a pharmaceutical composition, i.e. preferably is formulated as a medicament. A medicament in the sense of the present invention refers, preferably, to a pharmaceutical composition containing the biologically active CCR5 inhibitor as pharmaceutically active compound and one or more other components such as one or more pharmaceutically acceptable carrier(s). The pharmaceutically active compound can be present in liquid or lyophilized form. For example, the pharmaceutically active compound can be present together with glycerol and/or protein stabilizers (e.g., human serum albumin). The medicament is, typically, administered systemically and, preferably, intravenously or intramuscularly, or topically to the affected organ, in particular the lung. However, depending on the nature of the formulation and the desired therapeutic application, the medicament may be administered by other routes as well. The pharmaceutically active compound is the active ingredient or drug of the medicament, and is preferably administered in conventional dosage forms prepared by combining the drug with standard pharmaceutical carriers according to conventional procedures. These procedures may involve mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables. The carrier(s) must be acceptable in the sense of being compatible with the other ingredients of the formulation and being not deleterious to the recipient thereof. The pharmaceutical carrier employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington’s Pharmaceutical Sciences, Mack Publishing Company, Easton, Pennsylvania. The diluent(s) is/are selected so as not to affect the biological activity of the combination. Examples of such diluents are distilled water, physiological saline, Ringer’s solutions, dextrose solution, and Hank’s solution. In addition, the pharmaceutical composition or formulation may also include other carriers, adjuvants, or nontoxic, non-therapeutic, non-immunogenic stabilizers and the like. The medicament referred to herein is, preferably, administered at least once, e.g. as a bolus. However, the said medicament may be administered more than one time and, preferably, at least twice, e.g. permanently until improvement or periodically for defined time windows.

A therapeutically effective dose refers to an amount of the CCR5 inhibitor to be used in medicament which prevents, ameliorates or cures the symptoms accompanying a disease or condition referred to in this specification. Therapeutic efficacy and toxicity of a drug can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and by clinical factors. As is well known in the medical arts, dosages for any one patient depend upon many factors, including the patient’s size, age, the particular formulation of the medicament to be administered, sex, time and route of administration, general health, and other drugs being administered concurrently. Progress can be monitored by periodic assessment. Dosage recommendations shall be indicated in the prescribers or users instructions in order to anticipate dose adjustments depending on the considered recipient.

The medicament according to the present invention may in a further aspect of the invention comprise drugs in addition to the aforementioned compounds which are added during its formulation preferably as specified herein above. Preferably, the pharmaceutically active compound according to the invention is to be applied together with at least one further drug and, thus, may be formulated together with these other drugs as a medicament. More preferably, said at least one further drug is selected from the group consisting of: antibiotics, vasopressors, steroids, anticoagulants, antithrombotics, proinflammatory cytokines and DAMP inhibitors. Also, it is to be understood that the formulation of a pharmaceutical composition preferably takes place under GMP standardized conditions or the like in order to ensure quality, pharmaceutical security, and effectiveness of the medicament.

Preferred CCR5 inhibitors referred to herein are either approved for sale as pharmaceuticals or are actively developed for this purpose. Thus, the CCR5 inhibitors, their structures, their toxicities, and their galenic properties are in principle known to the skilled person and thus, preferably, a CCR5 inhibitor referred to herein is administered in the form, preparation, mode and/or dose as used for at least one known application of the compound.

Advantageously, it was found in the work underlying the present invention that CCR5 inhibitors are effective in treatment of coronavirus infections, in particular of SARS-CoV-2-associated pneumonia and sepsis.

As already outlined above, the immune modulator (preferably a CCR5 inhibitor) as defined above is particularly useful in medical settings.

Thus, in a preferred embodiment, the present invention relates to a pharmaceutical composition, comprising an effective amount of the immune modulator (preferably a CCR5 inhibitor) for use in treating and/or preventing pneumonia, sepsis and/or coronavirus infection according to the present invention as described above and at least one pharmaceutically acceptable excipient.

The terms “treatment” and/or “prevention” and the like have been defined above.

The pharmaceutical composition of the present invention may be administered via a large range of classes of forms of administration known to the skilled person. Administration may be systemically, locally, orally, through aerosols including but not limited to tablets, needle injection, the use of inhalators, creams, foams, gels, lotions and ointments.

In a preferred embodiment, the administration is orally.

An excipient or carrier is an inactive substance formulated alongside the active ingredient, i.e., the immune modulator (preferably a CCR5 inhibitor) as described above of the present invention for the purpose of bulking-up formulations that contain potent active ingredients. Excipients are often referred to as “bulking agents,” “fillers,” or “diluents”. Bulking up allows convenient and accurate dispensation of a drug substance when producing a dosage form. They also can serve various therapeutic-enhancing purposes, such as facilitating drug absorption or solubility, or other pharmacokinetic considerations. Excipients can also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors.

Thus, the pharmaceutical composition comprising an effective amount of the immune modulator (preferably a CCR5 inhibitor) of the present invention as described above may be in solid, liquid or gaseous form and may be, inter alia, in a form of (a) powder(s), (a) tablet(s), (a) solution(s) or (an) aerosol(s). It is preferred that said pharmaceutical composition optionally comprises a pharmaceutically acceptable carrier and/or diluent.

These pharmaceutical compositions can be administered to the subject at a suitable dose. Administration of the suitable compositions may be affected by different ways, e.g., by oral, intravenous, intraperitoneal, subcutaneous, intramuscular, topical, intradermal, intranasal or intrabronchial administration.

It is particularly preferred that said administration is carried out by oral administration.

It is particularly preferred that said administration is carried out by injection and/or delivery, e.g., to a site in a lung artery or directly into the lung. The compositions of the invention may also be administered directly to the target site, e.g., by biolistic delivery to an external or internal target site, like the lung.

The dosage regimen will be determined by the attending physician and clinical factors. As is well known in the medical arts, dosages for any one patient depends upon many factors, including the patient’s size, body surface area, age, the particular compound to be administered, sex, time and route of administration, general health, and other drugs being administered concurrently. Pharmaceutically active matter may be present in amounts between 1 ng and 10 mg/kg body weight per dose; however, doses below or above this exemplary range are envisioned, especially considering the aforementioned factors. If the regimen is a continuous infusion, it should also be in the range of 1 µg to 10 mg units per kilogram of body weight per minute.

In a preferred embodiment, the immune modulator (preferably a CCR5 inhibitor) is administered in the range of 1 mg to 500 mg, more preferably in the range of 1 mg to 400 mg, in the range of 1 mg to 300 mg or in the range of 1 mg to 200 mg per daily dose.

In a further preferred embodiment, the immune modulator (preferably a CCR5 inhibitor) is administered in the range of 10 mg to 500 mg, more preferably in the range of 50 mg to 500 mg, in the range of 100 mg to 500 mg or in the range of 150 mg to 500 mg per daily dose.

In further preferred embodiments, the immune modulator (preferably a CCR5 inhibitor) is administered at a daily dose of at most 500 mg, 450 mg, 400 mg, 350 mg, 300 mg, 250 mg, 200 mg, 150 mg, 125 mg, 100 mg or at most 50 mg.

In a more preferred embodiment, the immune modulator (preferably a CCR5 inhibitor) is administered at a daily dose of 150 mg. In an even more preferred embodiment, the immune modulator (preferably a CCR5 inhibitor) is administered as two doses, preferably of 75 mg per dose per day.

In an even more preferred embodiment, the above dosages relate to an oral administration. In a more preferred embodiment, in such a dosage regimen, the immune modulator is a CCR5 inhibitor, more preferably maraviroc.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose, i.e., in “an effective amount” which can easily be determined by the skilled person by methods known in the art. The dosage regimen will be determined by the attending physician and clinical factors. As is well known in the medical arts, dosages for any one patient depends upon many factors, including the patient’s or subject’s size, body surface area, age, the particular compound to be administered, sex, time and route of administration, general health, and other drugs being administered concurrently.

Thus, preferably, the immune modulator (preferably a CCR5 inhibitor) (and/or the “additional compound” (i.e., a compound against bacterial, fungal, and/or viral superinfections as described above) in case a combination therapy is envisaged) of the present invention as described above is included in an effective amount. The term “effective amount” refers to an amount sufficient to induce a detectable therapeutic response in the subject to which the pharmaceutical composition is to be administered. In accordance with the above, the content of the immune modulator (preferably a CCR5 inhibitor) of the present invention in the pharmaceutical composition is not limited as far as it is useful for treatment as described above, but preferably contains 0.0000001-10% by weight per total composition. Further, the immune modulator (preferably a CCR5 inhibitor) described herein is preferably employed in a carrier. Generally, an appropriate amount of a pharmaceutically acceptable salt is used in the carrier to render the composition isotonic. Examples of the carrier include but are not limited to saline, Ringer’s solution and dextrose solution. Preferably, acceptable excipients, carriers, or stabilisers are nontoxic at the dosages and concentrations employed, including buffers such as citrate, phosphate, and other organic acids; salt-forming counter-ions, e.g. sodium and potassium; low molecular weight (> 10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington’s Pharmaceutical Sciences, 17th ed., 1985, Mack Publishing Co.

Therapeutic progress can be monitored by periodic assessment. The immune modulator (preferably a CCR5 inhibitor) of the present invention or the pharmaceutical composition of the invention may be in sterile aqueous or non-aqueous solutions, suspensions, and emulsions as well as creams and suppositories. Examples of non-aqueous solvents are propylene glycol, polyethylene glycol, vegetable oils such as olive oil, and organic esters such as ethyl oleate. Aqueous carriers include water, alcoholic/aqueous solutions, emulsions or suspensions, including saline and buffered media. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition. Said agents may be, e.g., polyoxyethylene sorbitan monolaurate, available on the market with the commercial name Tween, propylene glycol, EDTA, Citrate, Sucrose as well as other agents being suitable for the intended use of the pharmaceutical composition that are well-known to the person skilled in the art.

In accordance with this invention, the term “pharmaceutical composition” relates to a composition for administration to a patient, preferably a human patient.

The definitions made above apply mutatis mutandis to the following. Additional definitions and explanations made further below also apply for all embodiments described in this specification mutatis mutandis.

The present invention further relates to a method of identifying a compound for treating and/or preventing coronavirus infection, pneumonia, and/or sepsis, comprising
a) contacting host cells comprising CCR5 with a candidate compound,
b) determining inhibition of CCR5 in said host cells, and
c) based on determination step b), identifying a compound for treating and/or preventing coronavirus infection, pneumonia, and/or sepsis.

The method of the present invention, preferably, is an in vitro method. Moreover, it may comprise steps in addition to those explicitly mentioned above. For example, further steps may relate, e.g., to providing host cells for steps a) further activity testing after step c). Moreover, one or more of said steps may be performed by automated equipment.

As used herein, the term “host cell” relates to any cell capable of maintaining and/or expressing, a CCR5 polypeptide. Preferably, the cell is a bacterial cell, more preferably a cell of a common laboratory bacterial strain known in the art, most preferably an Escherichia strain, in particular an E. coli strain. Also preferably, the host cell is an eukaryotic cell, preferably a yeast cell, e.g. a cell of a strain of baker’s yeast, or is an animal cell. More preferably, the host cell is an insect cell or a mammalian cell, preferably from a mammalian subject as specified herein above, in particular a mouse or rat cell. Most preferably, the host cell is a human cell. It is, however, also envisaged that the host cell is a plant cell. Preferred mammalian cell types are T-cells and macrophages.

In a more preferred embodiment, it also envisaged that preferred mammalian cell types are monocytes.

The term “candidate compound” is used herein in a broad sense relating to any chemical compound suspected to be a CCR5 inhibitor; thus, the term, preferably, relates to any compound not having previously been tested negative for CCR5 inhibitor activity. Methods for determining CCR5 inhibitor activity are known in the art and are described herein above.

The present invention also relates to a use of a CCR5 inhibitor of the present invention in the manufacture of a pharmaceutical composition for treating and/or preventing coronavirus infection, pneumonia, and/or sepsis in a subject.

The present invention also relates to a kit comprising a CCR5 inhibitor of the present invention and a means of administration of said compound to the lung.

The term “kit”, as used herein, refers to a collection of the aforementioned compounds, means or reagents which may or may not be packaged together. The components of the kit may be comprised by separate vials (i.e. as a kit of separate parts) or provided in a single vial. Moreover, it is to be understood that the kit of the present invention, preferably, is to be used for practicing the methods or uses referred to herein above. It is, preferably, envisaged that all components are provided in a ready-to-use manner for practicing the methods or uses referred to above. Further, the kit, preferably, contains instructions for carrying out said methods or uses. The instructions can be provided by a user’s manual in paper or electronic form. In addition, the manual may comprise instructions for administration and/or dosage instructions using the kit of the present invention.

Preferably, the kit comprises a means of administration of the compound to the lung. Preferred means for administration are those which can be applied without the particular knowledge of a specialized technician. Preferably, the means for administration is a lavage device. More preferably, the means for administration is an inhaler comprising the CCR5 inhibitor, wherein, more preferably, said compound is formulated for administration as an aerosol or as a dry powder, more preferably as an aerosol.

The present invention also relates to a device for administration of a compound to the lung, comprising a CCR5 inhibitor of the present invention.

The term “device”, as used herein, relates to a system of means comprising at least the aforementioned means operatively linked to each other as to allow the administration. Preferred devices have been described herein above as components of the kit.

The present invention also relates to a method of treating and/or preventing coronavirus infection, pneumonia, and/or sepsis in a subject, comprising contacting said subject with a CCR5 inhibitor, and thereby treating and/or preventing coronavirus infection, pneumonia, and/or sepsis.

As regards the preferred embodiments of the method for treatment and/or preventing the same applies, mutatis mutandis, as has been set forth above in the context of the immune modulator (preferably a CCR5 inhibitor) or the pharmaceutical composition for use as defined above.

In the present invention, the subject is, in a preferred embodiment, a mammal such as a dog, cat, pig, cow, sheep, horse, rodent, e.g., rat, mouse, and guinea pig, or a primate, e.g., gorilla, chimpanzee, and human. In a most preferable embodiment, the subject is a human.

In view of the above, the following embodiments termed items are particularly envisaged:
1. An immune modulator comprising
   (i) a compound of formula I or a pharmaceutically acceptable salt or solvate thereof: 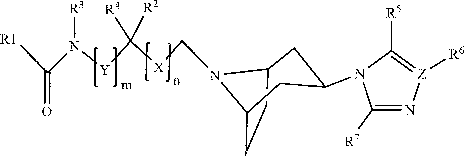
   wherein
      X and Y are independently selected from —CH₂— and —O—, preferably are —CH₂—;
      Z is N or C, preferably is N;
      m is an integer of from 0 to 3, preferably is 0;
      n is an integer of from 1 to 3, preferably is 1;
      R¹ is, optionally substituted, C₅-C₆ cycloalkyl or aryl;
      R² is, optionally substituted, aryl or heteroaryl;
      R³ and R⁴ are —H, or R³ and R⁴ together form a —CH₂—CH₂— bridge;
      R⁵ and R⁷ are independently selected from alkyl, preferably methyl and isopropyl; and
      in case Z is N, R⁶ is absent, or,
      in case Z is C, R⁶ together with R⁵ forms a —CH═CH—CH═CH— bridge; and/or
   (ii) a C—C chemokine receptor type 5 (CCR5) inhibitor,
      for use in treating and/or preventing pneumonia, sepsis, and/or coronavirus infection in a subject.
2. The immune modulator for use of item 1, wherein
   (i) M is 0;
   (ii) X is —CH2— and n is 1; \
   (iii) Z is N;
   (iv) R¹ is 4,4-difluoro-cyclohexanyl;
   (v) R² is aryl, preferably phenyl;
   (vi) R³ and R⁴ are —H; and/or
   (vii) R⁵ is methyl and R⁷ is isopropyl, preferably wherein said immune modulator is Maraviroc.
3. The immune modulator for use of item 1 or 2, wherein said CCR5 inhibitor is selected from the list consisting of Maraviroc, Vicriviroc, Aplaviroc, ancriviroc, Cenicriviroc, TAK-779, E913, TAK-220, AZD5672, BMS-681, CCR5 antagonist 34 , GSK2239633A, INCB009471, Leronlimab (PRO 140), viral macrophage inflammatory protein-II, and CCL7.
4. The immune modulator for use of item 1 or 2, wherein said CCR5 inhibitor is a low-molecular weight compound with a molecular mass of at most 1 kDa.
5. The immune modulator for use of any one of items 1 to 3, wherein said CCR5 inhibitor is selected from the list consisting of Maraviroc, Vicriviroc, and Aplaviroc.
6. The immune modulator for use of any one of items 1 to 5, wherein said compound of formula I is maraviroc.
7. The immune modulator for use of any one of items 1 to 6, wherein said coronavirus infection is coronavirus-associated pneumonia.
8. The immune modulator for use of any one of items 1 to 7, wherein said coronavirus infection is coronavirus-associated sepsis.
9. The immune modulator for use of any one of items 1 to 8, wherein said pneumonia is viral pneumonia.
10. The immune modulator for use of any one of items 1 to 9, wherein said pneumonia is coronavirus-associated pneumonia.
11. The immune modulator for use of any one of items 1 to 10, wherein said pneumonia is sepsis-associated pneumonia.
12. The immune modulator for use of any one of items 1 to 11, wherein said sepsis is pneumonia-associated sepsis.
13. The immune modulator for use of any one of items 1 to 12, wherein said sepsis is viral pneumonia-associated sepsis.
14. The immune modulator for use of any one of items 1 to 13, wherein said sepsis is coronavirus pneumonia-associated sepsis.
15. The immune modulator for use of any one of items 1 to 14, wherein said subject is a human.
16. The immune modulator for use of any one of items 1 to 15, wherein said subject is infected with a coronavirus.
17. The immune modulator for use of any one of items 1 to 16, wherein said subject suffers from coronavirus associated pneumonia and/or coronavirus associated sepsis.
18. The immune modulator for use of any one of items 1 to 17, wherein said treating and/or preventing comprises inhibition of monocyte migration to inflammation sites or wherein said treating and/or preventing comprises inhibition of T-cell migration to inflammation sites.
19. The immune modulator for use of any one of items 1 to 18, wherein said treating and/or preventing comprises reducing antigen presentation by antigen presenting cells (APC).
20. The immune modulator for use of any one of items 1 to 19, wherein said treating and/or preventing comprises inhibition of tissue destruction, preferably lung tissue destruction.
21. The immune modulator for use of any one of items 1 to 20, wherein said treating and/or preventing comprises inhibition of cell lysis.
22. The immune modulator for use of any one of items 1 to 21, wherein said treating and/or preventing comprises reducing or inhibiting an immune response, preferably inhibiting IL-6 and/or C-reactive protein and/or IL-12 and/or reactive oxygen species production.
23. The immune modulator for use of any one of items 1 to 22, wherein said treating and/or preventing comprises reducing the hypo-inflammatory phase of sepsis, in particular comprises increasing monocyte, preferably macrophage counts, at inflammation sites.
24. The immune modulator for use of any one of items 1 to 23, wherein said coronavirus is a beta-coronavirus.
25. The immune modulator for use of any one of items 1 to 24, wherein said coronavirus is severe acute respiratory syndrome coronavirus (SARS-CoV)-2, SARS-CoV-1, or Middle East respiratory syndrome coronavirus (MERS-CoV).
26. The immune modulator for use of any one of items 1 to 25, wherein said compound is comprised in a pharmaceutical composition.
27. The immune modulator for use of any one of items 1 to 26, wherein said pneumonia is human immunodeficiency virus (HIV)-associated pneumonia.
28. The immune modulator for use of any one of items 1 to 27, wherein said sepsis is HIV-associated sepsis.
29. The immune modulator for use of any one of items 1 to 28, wherein said HIV is an HIV using the CCR5 receptor as entry mediator.
30. A method of identifying a compound for treating and/or preventing coronavirus infection, pneumonia, and/or sepsis, comprising
   a) contacting host cells comprising CCR5 with a candidate compound,
   b) determining inhibition of CCR5 in said host cells, and
   c) based on determination step b), identifying a compound for treating and/or preventing coronavirus infection, pneumonia, and/or sepsis.
31. A use of a immune modulator as specified in any one of items 1 to 6 in the manufacture of a pharmaceutical composition for treating and/or preventing coronavirus infection, pneumonia, and/or sepsis in a subject.
32. A use of a immune modulator as specified in any one of items 1 to 6 in the manufacture of a pharmaceutical composition for treating and/or preventing coronavirus infection, pneumonia, and/or sepsis in a subject.
33. A kit comprising an immune modulator as specified in any one of items 1 to 6 and a means of administration of said compound to the lung.
34. A device for administration of a compound to the lung, comprising an immune modulator as specified in any one of items 1 to 6.

All references cited in this specification are herewith incorporated by reference with respect to their entire disclosure content and the disclosure content specifically mentioned in this specification.

### FIGURE LEGENDS

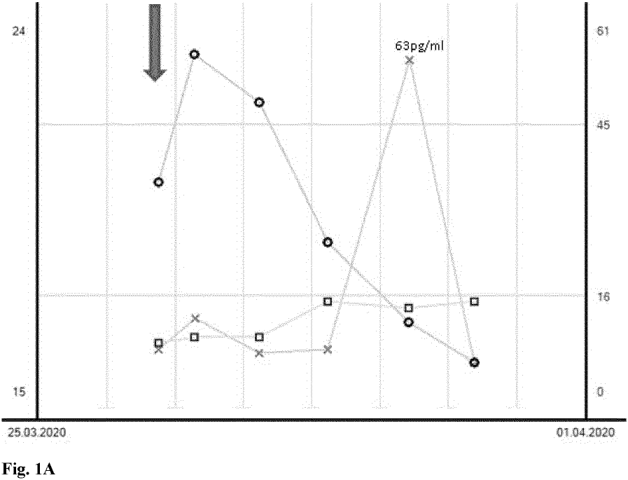

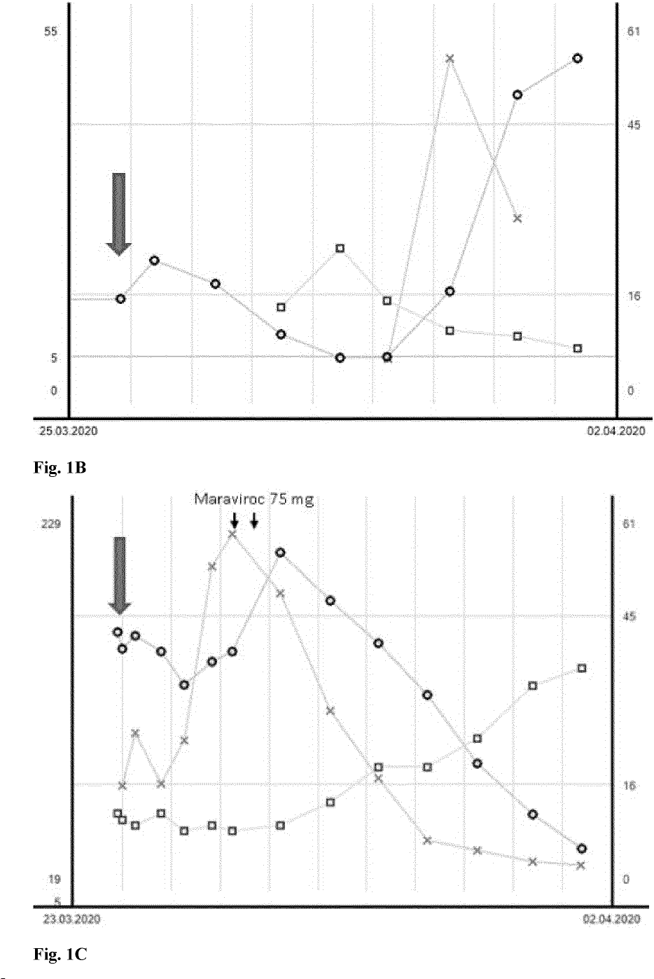

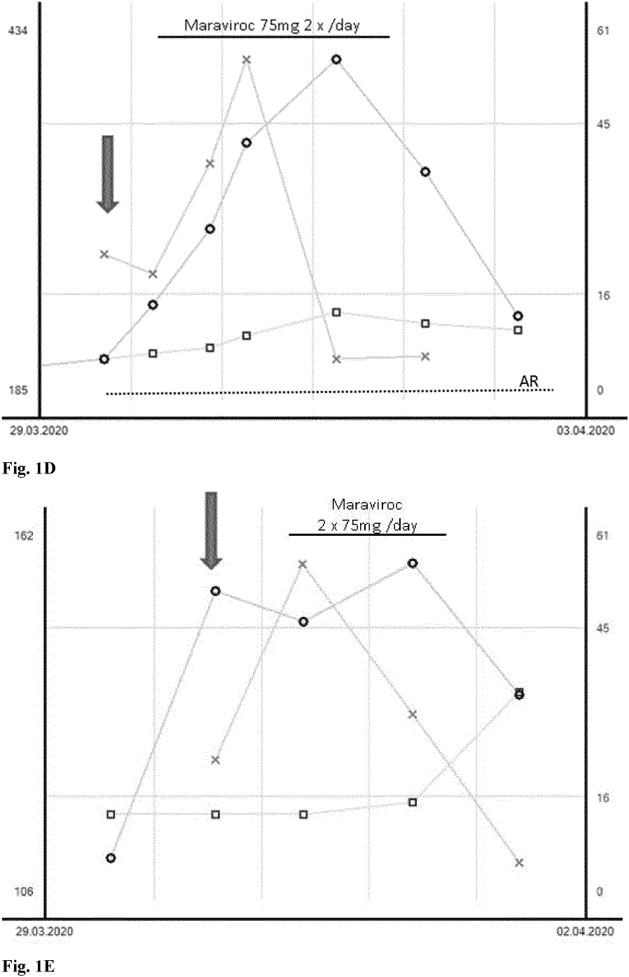

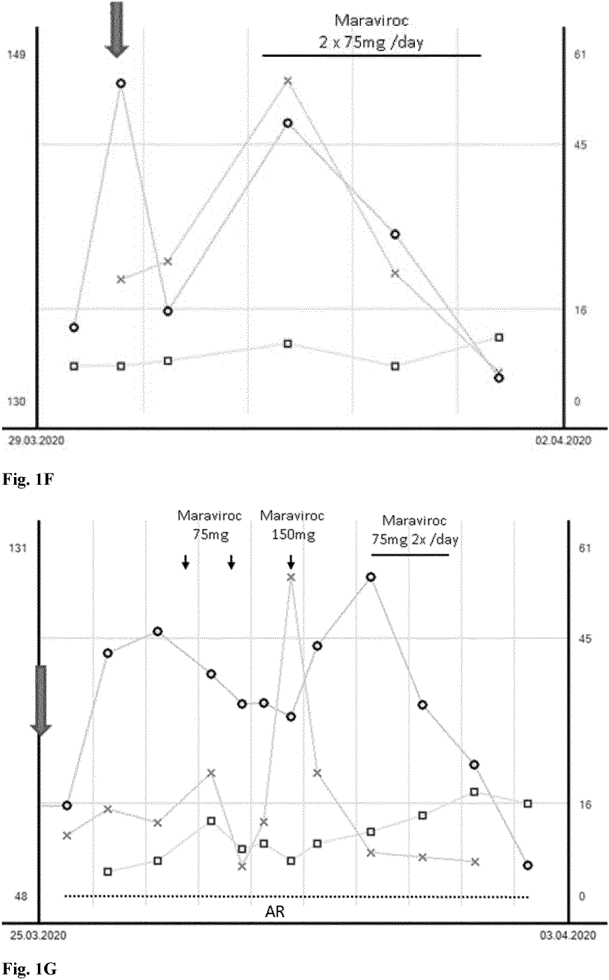

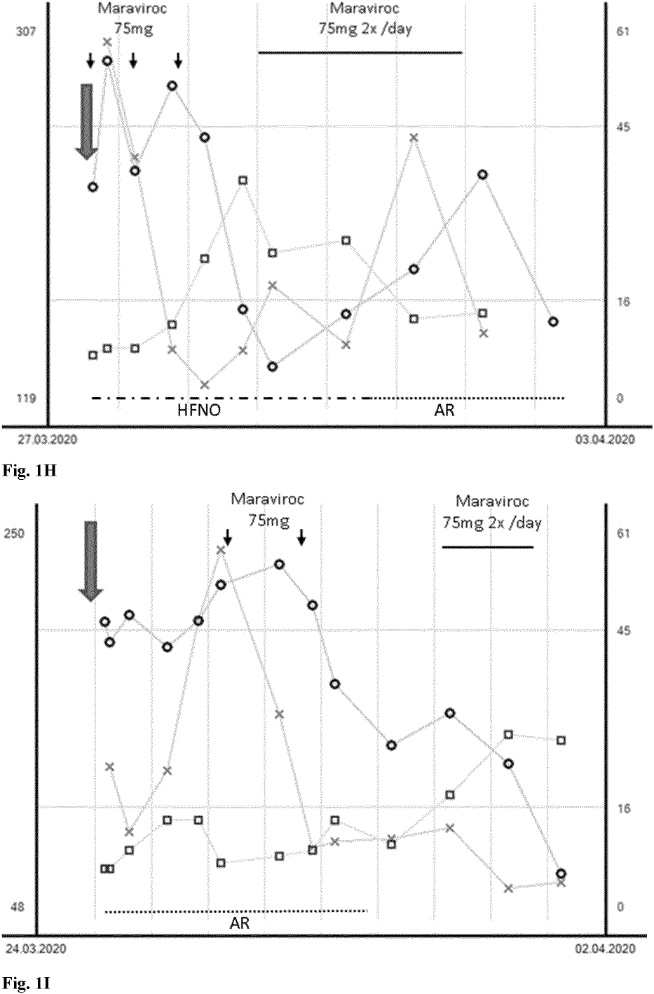

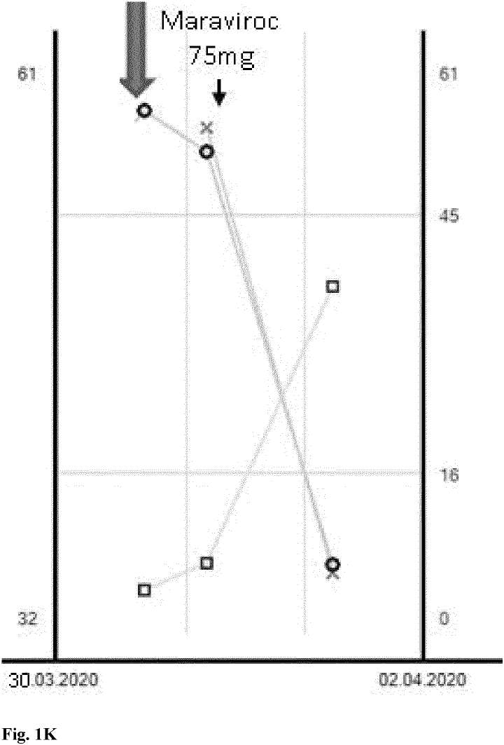

### FIG. 1: Time Course of Clinical Inflammation Parameters in Serum of Exemplary COVID-19 Pneumonia Patents

All patients received standard therapy with piperacillin/tazobactam, caspofungin, and hydroxychloroquine; in A) and B), patients did not receive further treatment, in C) to K), patients additionally received maraviroc (single doses (small arrows) or twice daily as indicated); circles and left y-axes: C-reactive protein (CRP) in mg/ml; rectangles and right y-axes: transferrin saturation in %; crosses: IL-6; AR and dotted lines: artificial respiration; HFNO: high-flow nasal oxygen; large arrows: start of standard treatment.

### FIG. 2: Course of Oxygen Requirements in Individual Patients From Baseline Until the End of the Observation

Each row represents one patient, numbered 1-47. Days are on the x-axis, starting at initiation day of maraviroc (day 1, baseline). The oxygen requirement of each patient on each day is represented by numbers on the 6-stage ordinal scale as indicated. Black vertical lines on any day depict the end of maraviroc treatment. If neither discharge nor death is noted, the patient was still on the respective ward on the last day of data acquisition. Improvement (light grey), no change (dark gray), or worsening (black) on the 6-stage ordinal scale from baseline until the last day are marked immediately to the left of each patient. On the far left, a dot indicates if that patient had received at least one dose of hydroxychloroquine (HCQ), tocilizumab, intravenous immunoglobulin (IVIG), prednisolone, underwent plasmapheresis, or if blood was analyzed for cytokine research during the observational period.

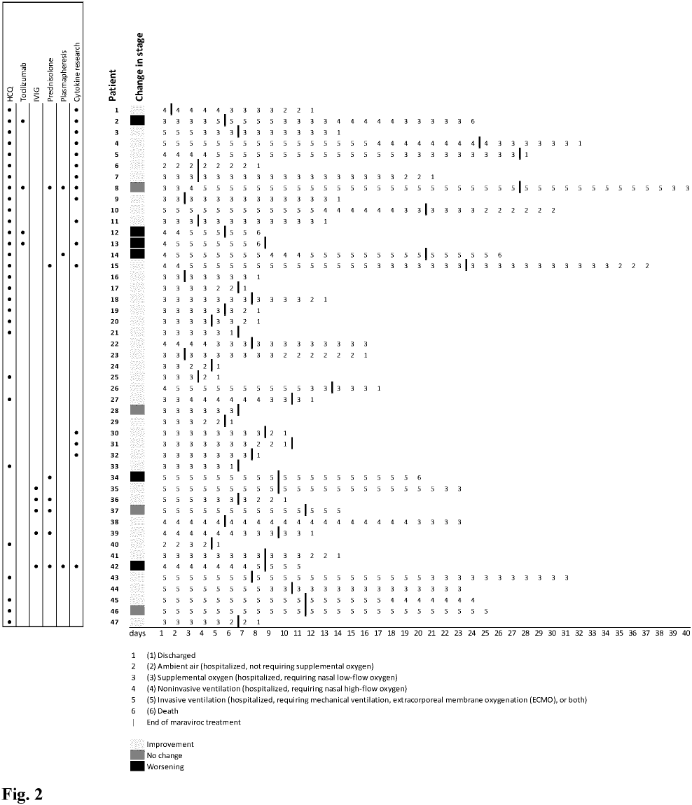

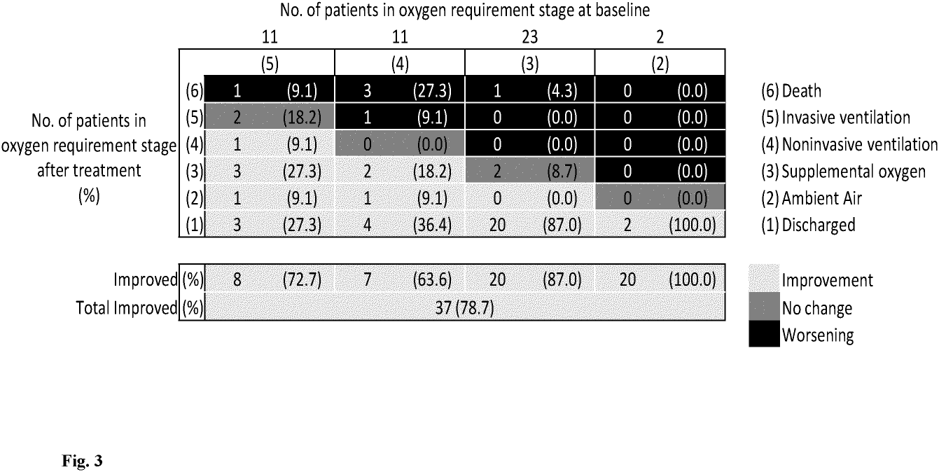

### FIG. 3: Cohort Oxygen Requirement at Baseline and After Treatment

Numbers of patients and their oxygen requirements are shown on the 6-stage ordinal scale, for time points baseline versus after treatment. After treatment is defined as either the last day of data acquisition, or discharge, or death, whichever occurred first. Percentages reflect how many patients who were in the specific stage changed their stage from baseline to after treatment.

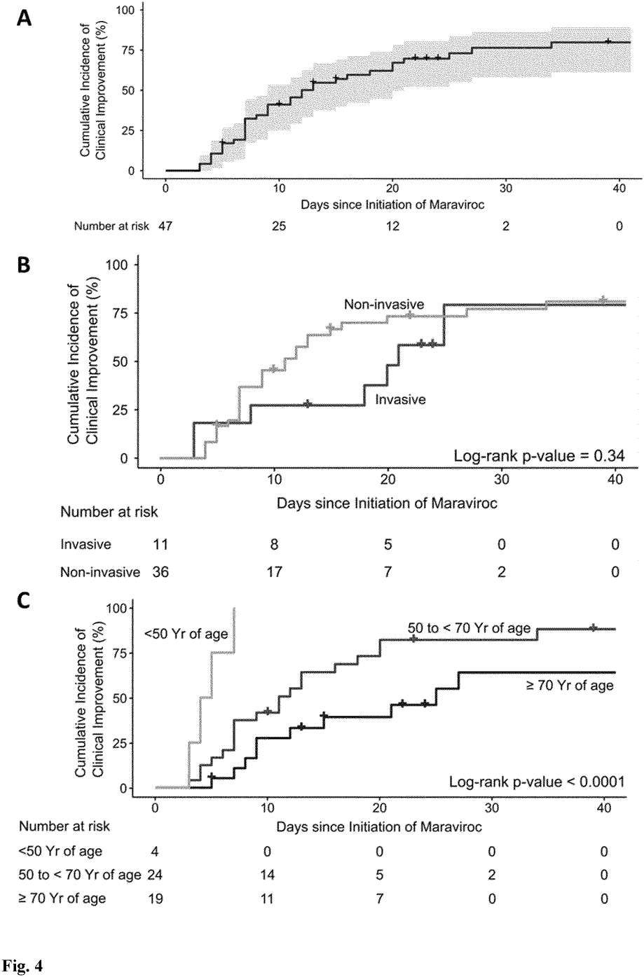

### FIG. 4: Cumulative Substantial Clinical Improvement From Baseline

Kaplan-Meier analyses of the cumulative substantial clinical improvement in percent, including number at risk. A decrease of two stages or more on the ordinal scale or a discharge (whichever occurred first) are defined as a substantial clinical improvement. Deceased patients are included as non-improvement until the end of the study. All panels show substantial clinical improvement over days post-baseline and are stratified as follows: (A) Overall cohort; (B) stratified by baseline oxygen support: invasive mechanical ventilation versus no invasive mechanical ventilation; and (C) stratified by age. <50 years of age, 50 to <70 years of age, ≥70 years of age. The oldest group compared to the youngest group shows a significant difference (hazard ratio of 0.12, 95% CI (0-0.2), p-value<0.0001)

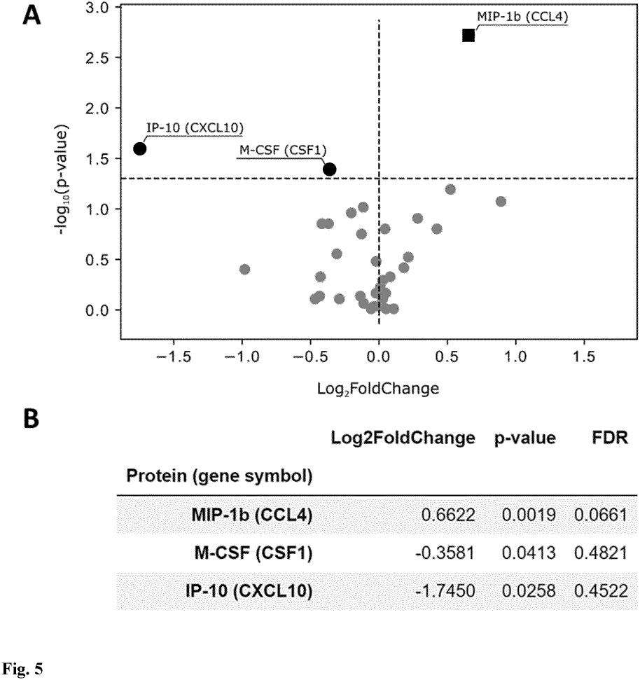

### FIG. 5: Observed Cytokine Changes From Baseline

(A) Volcano plot showing differential cytokine expression from blood samples before and after maraviroc treatment. Significantly increased proteins with their gene symbols are highlighted as black squares and decreased proteins as black dots, all other proteins are shown in gray. After maraviroc treatment is defined as 5 days or more after baseline. (B) Shows a table describing the most significantly changed proteins, their log₂-fold change, p-values, and false discovery rate (FDR).

The following Examples shall merely illustrate the invention. They shall not be construed, whatsoever, to limit the scope of the invention.

### Example 1

Surprisingly, it was found that patients suffering from a severe course of COVID-19 had increased levels of RANTES (CCL5), a ligand of CCR5.

Patients with diagnosed SARS-CoV-2 pneumonia received standard therapy with piperacillin/tazobactam, caspofungin, and hydroxychloroquine; where deemed clinically necessary, additionally Maraviroc, a CCR5 inhibitor, was administered as indicated in FIG. 1.

Patient characteristics were as follows:
Patient A (FIG. 1A): male, 38 yrs, no relevant previous disease;
Patient B (FIG. 1B): male, 38 yrs;
Patient C (FIG. 1C): male, 66 yrs, no relevant previous disease;
Patient D (FIG. 1 D): male, 58 yrs, state after chemotherapy for Burkitt lymphoma; under artificial respiration;
Patient E (FIG. 1E): male, 72 yrs;
Patient F (FIG. 1 F): male, 63 yrs;
Patient G (FIG. 1G): female, 77 yrs, under artificial respiration;
Patient H (FIG. 1 H):male, 78 yrs;
Patient I (FIG. 1 I): male 41 yrs;
Patient K (FIG. 1 K): male, 64 yrs.

FIG. 1 shows a time course of clinical inflammation parameters in serum of exemplary COVID-19 pneumonia patents. All patients received standard therapy with piperacillin/tazobactam, caspofungin, and hydroxychloroquine; in A) and B), patients did not receive further treatment, in C) to K), patients additionally received maraviroc (single doses (small arrows) or twice daily as indicated); circles and left y-axes: C-reactive protein (CRP) in mg/ml; rectangles and right y-axes: transferrin saturation in %; crosses: IL-6; AR and dotted lines: artificial respiration; HFNO: high-flow nasal oxygen; large arrows: start of standard treatment.

As can be derived from the data presented in FIG. 1, increase of inflammation markers could be reverted in a very fast manner in all cases after administration of Maraviroc; in some cases, repeated administration was necessary.

### Example 2: Further Analysis of Cases of Maraviroc Investigational Therapy For Patients With Severe Covid-19

### Methods

### Data Acquisition and Analysis

The presented severe COVID-19 cases were documented during investigational therapy with maraviroc at the Heidelberg University Hospital, Germany. Treatment and follow up took place between Mar. 26, 2020 and the last day of data acquisition, May 7, 2020. Follow up took place until discharge, death, or the last day of data acquisition, whichever occurred first. As the treatment was given on an individual per patient basis without a control cohort, no sample-size calculations, trial protocol, or endpoints were pre-defined. Data analysis was approved by the Ethics Committee of the Medical Faculty Heidelberg.

### Patients

Criteria for initiating investigational maraviroc therapy were laboratory-confirmed SARS-CoV-2 infection, age of 18 years or older, hospitalization of the patient due to severe COVID-19, and voluntary informed consent. Diagnosis of SARS-CoV-2 infection was based on a positive quantitative polymerase chain reaction to detect the viral genome from respiratory tract samples. Severe COVID-19 was defined as patients showing one or more of the following characteristics at admission to hospital: Respiratory rate ≥30/min, blood oxygen saturation ≤93%, ratio of partial pressure of oxygen in arterial blood over the fraction of inspired oxygen <300.

Maraviroc was initially used if severely ill patients showed no improvement after approximately three days of antibiotic therapy. Later, this time period was shortened for several patients due to observed signs of efficacy. Maraviroc was not used if patients had a known allergy to maraviroc, were pregnant, or were breastfeeding.

Patients for whom progression to death was imminent and inevitable within the next 24 hours, irrespective of the provision of treatments, and also patients who had given a do-not-resuscitate-and-intubate order could not be included in the analysis.This order limits ventilation options that are a core component of care for COVID-19 patients, and thus substantially influences the outcome.

### Treatment

The median daily dose of maraviroc per patient from baseline (first day of maraviroc treatment) until the end of treatment was 150 mg (interquartile range (IQR) 138-150 mg), including very few exceptions where the dose was omitted (0 mg).

Maraviroc was orally administered, generally as two doses of 75 mg each per day for a median of 7 days (IQR 5-10 days).

Additional therapeutic substances that were used are listed in the following. As prophylaxis and/or treatment of bacterial, fungal, or viral superinfections, 44 of 47 patients (93.6%) received a combination of piperacillin/tazobactam, generally with caspofungin.

Additionally, the following number of patients (out of 47) received at least one dose of meropenem (7), azithromycin (11), vancomycin (5), co-trimoxazole (2), ampicillin/sulbactam (1), ceftriaxone (1), flucloxacilin (1), moxifloxacin (1), aciclovir (7), ganciclovir (2), or voriconazole (1). Additional substances or procedures that the following number of patients received, including experimental therapies, included hydroxychloroquine (HCQ) (29), prednisolone (7), intravenous immunoglobulin (IVIG) (5), tocilizumab (4), and plasmapheresis (3). Supportive care and symptom management was carried out at the discretion of the treating physician.

### Outcome Measures

The description of the clinical course and outcome measures were based on change in oxygen requirements, discharge, or death.

A 6-stage ordinal scale was applied, modified from scales commonly used in influenza and COVID-19 studies (Grein J, Ohmagari N, Shin D, et al., N Engl J Med 2020; Davey RT, Jr., Fernandez-Cruz E, Markowitz N, et al., Lancet Respir Med 2019;7:951-63; Wang Y, Fan G, Salam A, et al., J Infect Dis 2019; Cao B, Wang Y, Wen D, et al., N Engl J Med 2020), defined as follows: (1) Discharged; (2) Ambient air (hospitalized, not requiring supplemental oxygen); (3) Supplemental oxygen (hospitalized, requiring nasal low-flow oxygen); (4) Noninvasive ventilation (hospitalized, requiring nasal high-flow oxygen); (5) Invasive ventilation (hospitalized, requiring mechanical ventilation, extracorporeal membrane oxygenation (ECMO), or both); (6) Death.

In addition to analyzing these precise changes, and especially to allow for improved comparability with studies which in several cases apply a second set of criteria, more substantial changes in clinical condition were analyzed with the following criteria: a decrease of two stages or more on the ordinal scale, or a discharge (whichever occurred first), were considered a substantial clinical improvement, and worsening was defined as an increase of at least one stage.

### Clinical and Research Laboratory Analysis

Clinical laboratory parameters were monitored daily. If patients gave voluntary consent, additional blood for cytokine and chemokine profiling in a research context was obtained and stored at -80° C. until use in the multiplex analysis (Bio-Plex Pro Human Cytokine 48-Plex Screening Panel plus ICAM and VCAM, Biorad, Munich, Germany) as described previously (Halama N, Michel S, Kloor M, et al., Cancer Res 2011;71:5670-7). Median protein concentrations per patient were compared in two groups: before and five or more days after the first day of maraviroc treatment.

### Statistical and Computational Analysis

For research cytokine analyses, cytokines which were undetectable in more than 3 samples were excluded. Correction for batch effects from three experiments was performed using the Python packagepycombat (version 0.14), and was validated by analyzing the mixture of samples within UMAP embeddings (McInnes L, Healy J, Melville J. UMAP: Uniform Manifold Approximation and Projection for Dimension Reduction. arXiv 2018). Wilcoxon signed-rank tests were used when comparing cytokine concentrations between groups. Resulting p-values were adjusted for multiple hypothesis testing according to the Benjamini and Hochberg method (Benjamini Y, Hochberg Y., J R Stat Soc B 1995;57:289-300). Kaplan-Meier estimator of survival was employed to visualize cumulative clinical improvement. Hazard ratio and the logrank test were used to compare improvement between patients in different groups using the R packages survival, survminer, and cmprsk. Stated percentages and quartiles include rounded numbers. Statistical analyses were carried out using R-3.5.3, Python-3.6.4, Excel (Microsoft), and SigmaPlot (Systat Software, Inc).

### Results

### Patients

The results are based on 47 retrospectively analyzed cases treated with maraviroc and subsequent follow up. From baseline (first maraviroc treatment) and within the 40 day observational period, the median follow-up duration was 12 days (interquartile range (IQR) 7-22 days).

### Patient Numbers and Characteristics at Baseline

Patient demographic and clinical characteristics at baseline (initiation day of maraviroc treatment) are shown in Table 1.

Patients were between 26 and 87 years old (median 64 years, IQR 58-76 years) and 34 patients (72.3%) were male. 45 patients (95.7%) required oxygen support at baseline, with 11 (23.4%) requiring invasive ventilation.

Stratifying the cohort into two subgroups, one receiving invasive mechanical ventilation and one not receiving invasive mechanical ventilation (receiving non-invasive oxygen support or breathing ambient air), a few differences were observed. The invasively ventilated group had a slightly higher median age than those without invasive ventilation (68 vs. 64 years), showed higher median levels of alanine aminotransferase (ALT) (50 vs. 35.5 IU per liter), aspartate aminotransferase (AST) (93 vs. 41.5 IU per liter), creatinine (1.29 vs. 0.89 mg per deciliter), C-reactive protein (CRP) (187 vs. 127 mg per liter), white blood count (11.69 vs. 7.16 per nanoliter), and substantially higher interleukin 6 (IL-6) (157 vs. 69.2 pg per milliliter). The two groups showed some differences in coexisting conditions, however no striking differences were observed.

TABLE 1

Patient Baseline Demographic and Clinical Characteristics
Number of patients

| Total number (N =) | Invasive mechanical ventilation 11 | | | Non-invasive 34 | | | Total 47 | | |
|---|---|---|---|---|---|---|---|---|---|
| Characteristic | | | | | | | | | |
| Median age - yr (IQR) | 68 | 58 | 76 | 64 | 57 | 77 | 64 | 58 | 76 |
| Age category - no. (%) | | | | | | | | | |
| <50 yr | 1 | 9.09 | | 3 | 8.57 | | 4 | 8.51 | |
| 50 to <70 yr | 5 | 45.45 | | 18 | 51.43 | | 24 | 51.06 | |
| ≥70 yr | 5 | 45.45 | | 14 | 40.00 | | 19 | 40.43 | |
| Male sex - no. (%) | 8 | 72.73 | | 26 | 72.73 | | 34 | 72.34 | |
| Oxygen requirement stage - no. (%) | | | | | | | | | |
| (5) Invasive ventilation | 11 | 23.40 | | | | | 11 | 23.40 | |
| (4) Noninvasive ventilation | | | | 11 | 23.40 | | 11 | 23.40 | |
| (3) Supplemental oxygen | | | | 23 | 48.94 | | 23 | 48.94 | |
| (2) Ambientair | | | | 2 | 4.26 | | 2 | 4.26 | |
| Median duration of symptoms befor emaraviroc therapy - days (IQR) | 15 | 10 | 22 | 10 | 7 | 15 | 12 | 8 | 15 |
| Coexisting conditions - no. (%) | | | | | | | | | |
| Any condition | 9 | 81.82 | | 24 | 70.59 | | 33 | 70.21 | |
| Arterial hypertension | 5 | 45.45 | | 18 | 52.94 | | 23 | 48.94 | |
| Coronary heart disease | 4 | 36.36 | | 7 | 20.59 | | 11 | 23.40 | |
| COPD | 2 | 18.18 | | 2 | 5.88 | | 4 | 8.51 | |
| Asthma | 1 | 9.09 | | 2 | 5.88 | | 3 | 6.38 | |
| Interstitial lungdisease | 0 | 0.00 | | 2 | 5.88 | | 2 | 4.26 | |
| Malignant disease | 1 | 9.09 | | 5 | 14.71 | | 6 | 12.77 | |
| Stroke | 0 | 0.00 | | 1 | 2.94 | | 1 | 2.13 | |
| Type 2 diabetes | 3 | 27.27 | | 5 | 14.71 | | 8 | 17.02 | |
| Autoimmune disease | 0 | 0.00 | | 1 | 2.94 | | 1 | 2.13 | |
| Obesity | 3 | 27.27 | | 1 | 2.94 | | 4 | 8.51 | |
| Premedication | | | | | | | | | |
| ACEI | 1 | 9.09 | | 8 | 23.53 | | 9 | 19.15 | |
| AT1R-Antagonist | 1 | 9.09 | | 4 | 11.76 | | 5 | 10.64 | |
| Chemotherapy within past4weeks | 1 | 9.09 | | 0 | 0.00 | | 1 | 2.13 | |
| NSAID within past 4 weeks | 1 | 9.09 | | 3 | 8.82 | | 4 | 8.51 | |
| Immunmodulation | 0 | 0.00 | | 4 | 11.76 | | 4 | 8.51 | |
| Median laboratory values (IQR) | | | | | | | | | |
| ALT- IU perliter | 50 | 38 | 77 | 35.5 | 25.25 | 51 | 38 | 27 | 57 |
| AST- IU per liter | 93 | 59 | 125 | 41.5 | 33.25 | 69.75 | 52 | 35 | 93 |
| Creatinine - mg per deciliter | 1.29 | 0.69 | 4.02 | 0.89 | 0.7625 | 1.0875 | 0.91 | 0.725 | 1.29 |
| CRP - mg per liter | 187 | 59 | 264 | 127 | 59 | 153 | 130 | 63 | 164 |
| White blood count - per nanoliter | 11.69 | 6.90 | 15.57 | 7.16 | 5.26 | 8.76 | 7.62 | 5.32 | 11.45 |
| Lymphocytes absolute - per nanoliter | 0.68 | 0.50 | 1.07 | 0.82 | 0.64 | 1.03 | 0.80 | 0.59 | 1.03 |
| Interleukin 6 - pg per mililiter | 157 | 17 | 304 | 69.2 | 30.075 | 102.5 | 73.3 | 30.075 | 125.5 |

Abbreviations: IQR -interquartile range; COPD - chronic obstructive pulmonary disease; HIV - human immunodeficiency virus; ACEI - Angiotensin-converting enzyme inhibitor; AT1R -Angiotensin II receptor type 1; NSAID - non-steroidal anti-inflammatory drug; ALT - alanine aminotransferase; AST - aspartate aminotransferase; CRP - C-reactive protein; IU -international units; no. - number. To convert creatinine mg/deciliter to micromoles/liter, multiply by 88.42.

### Oxygen Requirements and Clinical Improvement During Maraviroc Treatment

Changes in condition on the 6-stage ordinal scale for each patient over time are depicted in FIG. 2 and are summarized in FIG. 3. 37 of 47 patients (78.7%) showed an improvement on the 6-stage ordinal scale from baseline until the last day of data acquisition, and 29 patients (61.7%) could be discharged. 4 patients (8.5%) showed no change and 6 (12.8%) worsened, including 5 deaths (11%). Improvement on the 6-stage ordinal scale was observed in 8 of 11 patients (72.7%) receiving invasive ventilation, in 7 of 11 patients (63.6%) receiving non-invasive ventilation, in 20 of 23 patients (87%) receiving supplemental low-flow oxygen, and in 2 of 2 patients (100%) breathing ambient air at baseline.

Stratifying the cohort into the two subgroups, one receiving invasive mechanical ventilation and one not receiving invasive mechanical ventilation (all others), of 11 patients receiving invasive ventilation at baseline, 8 (72.7%) showed an improvement, 2 (18.2%) remained under invasive ventilation, and 1 (9.1%) worsened (died).

In the subgroup without invasive mechanical ventilation at baseline, 29 of 36 patients (80.6%) improved, 2 of 36 patients (5.6%) showed no change, and 5 of 36 patients (13.9%) worsened. Overall, for patients who improved on the ordinal scale, improvement took place in median 8 days (IQR 5-13 days) from baseline. For the group receiving invasive ventilation at baseline, improvement took place in median after 14 days (IQR 5-19 days) and for patients not receiving invasive ventilation, after a median of 7 days (IQR 5-11 days).

Though confounding factors such as concomitant medication cannot be excluded, patients treated with at least one dose of HCQ with those that did not were compared, and found that (i), the groups showed only minor differences in distribution of oxygen requirement stages at baseline (20.7% of patients vs. 27.8% in stage 5; 20.7% vs. 27.8% in stage 4; 51.7% vs. 44.4% in stage 3; and 6.9% vs. 0% in stage 2, respectively), (ii), the groups had no substantial difference in median time to improvement (6 days [IQR 3-13 days] vs. 7 days [IQR 3-11 days], respectively), and (iii), 75.9% (22 of 29) vs. 77.9% (14 of 18) showed an improvement during the observational period.

Additional concomitant investigational treatments were administered to substantially fewer patients and cannot be stratified as above. The number of patients who, before or concomitant to receiving maraviroc, also received at least one dose or procedure of the following are listed here, including their change on the ordinal scale from baseline during the observational periods (also see FIG. 1): prednisolone (3 of 7 improved, 2 showed no change, 2 worsened, 1 of which died); intravenous immunoglobulin (IVIG) (3 of 5 improved, 1 showed no change, 1 worsened), tocilizumab (1 of 4 showed no change, 3 worsened and died), and plasmapheresis (1 of 3 showed no change, 2 worsened, 1 of which died).

As described in the methods section, in addition to analyzing the precise clinical course on the 6-stage ordinal scale (FIG. 2 and FIG. 3), more substantial changes in clinical condition with the following criteria were also analyzed: a decrease of two stages or more on the 6-stage ordinal scale or a discharge, whichever occurred first, are considered a substantial clinical improvement. Worsening was defined as an increase of at least one stage. On the day of final data acquisition, a total of 72% of patients (34 of 47) had shown a substantial improvement in clinical condition.

The median time to a substantial clinical improvement from baseline was 9 days (IQR 7-16 days). The cumulative substantial changes in clinical condition are depicted in Kaplan-Meier plots in FIG. 4.

A higher percentage of patients showed clinical improvement if they were in the younger group (under 50-year olds vs. over-70 year olds) (hazard ratio of 0.12 (95% confidence interval (CI), 0-0.2, p-value<0.0001) (FIG. 4C).

### Cytokine Measurements

In our patient cohort, during clinical laboratory measurements, we observed elevated IL-6 levels at baseline (median 74.7 pg per milliliter), a higher median IL-6 level in the invasively ventilated versus the non-invasive group (157 vs. 69.2 pg per milliliter), and a statistically significant (p-value=0.002) overall decrease from the 74.7 pg per milliliter, to a median of 37.7 pg per milliliter three days after administration of maraviroc. The analyzed data contain very few data gaps (for example; at baseline, IL-6 laboratory values were available for 41 of 47 patients). Further research serum cytokine and chemokine analyses of a subset of 14 patients showed a significant increase in concentration of MIP-1b (CCL4) from before to after treatment with maraviroc (FIG. 5). A decrease in IP-10 (CXCL10) and M-CSF (CSF1) with p-values <0.05 was also observed, however due to the small sample size cannot be confirmed as significant after adjustment for multiple testing (FIG. 5).

### Safety, Mortality, and Laboratory Parameters

During the severe course of COVID-19 and after or during maraviroc treatment, one patient (2.1%) worsened from non-invasive to invasive ventilation and 5 patients (11%) died (FIG. 2). The five patients who died were 59, 71, 79, 82, and 84 years of age.

Patient 43 showed a strong increase in markers of liver function on day 5, namely a 2.8-fold increase in ALT and a 3.6-fold increase in AST from baseline, which both decreased on days 6 and 7. As defined by the Common Terminology Criteria for Adverse Events (CTCAE) v5.0, this is a grade 2, moderate liver function AE. Monitoring of clinical laboratory parameters also included, among others, serum levels of creatinine for kidney function, white blood count, lymphocyte count, and interleukin 6 (IL-6) as markers of inflammation and infection. Clinical parameters were monitored daily by clinicians and no other clinically significant changes were observed.

### Summary

There are currently no treatments against COVID-19 proven effective, merely a very limited number of substances with emergency use authorizations or approval in few countries. In parallel to endeavors aiming to develop vaccines, studies will need to address treatment, especially of severe COVID-19. The presented data on investigational use of maraviroc for treatment of severe COVID-19 shows an improvement on the 6-stage ordinal scale in 37 of 47 patients (78.7%) and a substantial clinical improvement (two or more stages improved or discharge) in 34 of 47 patients (72%).

The median time between symptom onset and maraviroc treatment begin, as well as the median time to improvement on the ordinal scale was substantially shorter for patients not receiving invasive ventilation at baseline (10 days after onset, 7 days to improvement) than for patients receiving invasive ventilation at baseline (15 days after onset, 14 days to improvement).

The above analyses show that the groups that received or did not receive HCQ had a similar distribution of oxygen requirements at baseline, had highly similar median times to improvement, and highly similar percentage of patients improved.

Two trials (Halama N, Zoernig I, Berthel A, et al., Cancer Cell 2016;29:587-601; Reshef R, Luger SM, Hexner EO, et al., N Engl J Med 2012;367:135-45) independently showed the safe use of maraviroc in addition to its use as an HIV drug.

In the present study, no novel AEs have been observed. Due to frequent liver comorbidities and frequently elevated levels of ALT and AST in COVID-19 (Zhang C, Shi L, Wang FS., Lancet Gastroenterol Hepatol 2020;5:428-30), it has not been differentiated whether the liver-specific AE was caused by COVID-19, maraviroc, underlying diseases, or other factors.

Several trials have been conducted in the search for COVID-19 treatments. A recent trial reported first data on a lack of benefit beyond standard care of lopinavir-ritonavir for adult patients with severe COVID-19 (Cao B, Wang Y, Wen D, et al., N Engl J Med 2020).

Another study presented data on compassionate use of remdesivir, which may show a benefit for severe cases (Grein J, Ohmagari N, Shin D, et al., N Engl J Med 2020). The remdesivir study showed an improvement in 36 of 53 patients (68%) in oxygen support and a worsening in 8 of 53 patients (15%), on a 6-point ordinal scale, highly similar to our scale, and a mortality of 13% over a median follow-up of 18 days. The demographic and clinical characteristics of this patient cohort differ from ours, including a higher proportion of patients requiring invasive ventilation at baseline in the remdesivir study.

Other studies have reported mortalities of 50% (12 of 24) of critically ill COVID-19 patients within an 18 day period (Bhatraju PK, Ghassemieh BJ, Nichols M, et al., N Engl J Med 2020), 21.9% (44 of 201) of hospitalized COVID-19 patients during a median hospital stay of 13 days (IQR 10-16 days) (Wu C, Chen X, Cai Y, et al., JAMA Intern Med 2020), 19.2% or 25% of severely ill COVID-19 patients over a 28 day period (with or without lopinavir-ritonavir, respectively, in two cohorts with a total of 199 patients together) (Cao B, Wang Y, Wen D, et al., N Engl J Med 2020), 15% (6 of 41; this cohort had a comparably low median age of 49 years, and presumably substantially less severely ill patients, as only 4 patients required invasive ventilation during the observational period between Dec. 16, 2019 and Jan. 22, 2020) (Huang C, Wang Y, Li X, et al., Lancet 2020;395:497-506), 11% (11 of 99) (this cohort had a comparably low median age of 55.5 years, and presumably substantially less severely ill patients, as merely 4 patients required invasive ventilation during the 25-day observational period) (Chen N, Zhou M, Dong X, et al., Lancet 2020;395:507-13), 10.2% (40 of 393) over a 36-day observational period (an early intubation strategy was followed) (Goyal P, Choi JJ, Pinheiro LC, et al., N Engl J Med 2020), and 48.5% (2497 of 5139) of patients that were in critical care during an observational period of approximately 60 days (center i-icnar. ICNARC report on COVID-19 in critical care - 01 May 2020. wwwicnarcorg 2020).

Herein above, an overall mortality of 11% from baseline to last day of data acquisition (40 day time frame with a median follow-up of 12 days) is reported. Study results are strongly dependent on demographic characteristics of each cohort, follow-up period, details in definition of severity, timely hospitalization, availability of medical measures, and several additional factors.

For patients who required invasive mechanical ventilation, recently published studies have reported different mortalities, ranging from 97% (31 of 32) (Zhou F, Yu T, Du R, et al., Lancet 2020;395:1054-62.), 67% mortality in 15 patients (Arentz M, Yim E, Klaff L, et al., JAMA 2020), 65.7% (44 of 67) (Wu C, Chen X, Cai Y, et al., JAMA Intern Med 2020), 14.6% (19 of 130 with an early intubation strategy) (Goyal P, Choi JJ, Pinheiro LC, et al., N Engl J Med 2020), to 18% (6 of 34) in the afore mentioned remdesivir study (Grein J, Ohmagari N, Shin D, et al., N Engl J Med 2020).

In the present hereinabove analysis, a 9% mortality (1 of 11) in severely ill patients was reported that were mechanically ventilated at baseline and treated with maraviroc (40 day observational period with a median follow-up of 12 days).

Two trials (Halama N, Zoernig I, Berthel A, et al., Cancer Cell 2016;29:587-601; Reshef R, Luger SM, Hexner EO, et al., N Engl J Med 2012;367:135-45) independently corroborated the modulatory properties of CCR5 inhibition in different clinical settings apart from HIV, namely in graft versus host disease and colorectal cancer. The inflammatory state in graft versus host disease is characterized by an unwanted influx, activation, and expansion of autoreactive T cells. The inhibitory effect that CCR5 has on macrophages, especially under the perspective of antigen presenting cells, is not yet comprehensively characterized. However, the capacity of CCR5 inhibition in activating macrophages is well characterized (Halama N, Zoernig I, Berthel A, et al., Cancer Cell 2016;29:587-601) and also described in the context of antigen processing (Schiavo R, Baatar D, Olkhanud P, et al., Blood 2006;107:4597-605).

Hereinabove, a significant increase of MIP-1b (CCL4), as well as a decrease of M-CSF (CSF1) and IP-10 (CXCL10) from before to after maraviroc treatment was observed.

IP-10 (CXCL10) has been suggested to be significantly elevated in severe COVID-19 cases (Huang C, Wang Y, Li X, et al., Lancet 2020;395:497-506; Xiong Y, Liu Y, Cao L, et al., Emerg Microbes Infect 2020;9:761-70), to be predictor of progression, and continuous high levels may be associated with disease deterioration and fatal outcome (Yang Y, Shen C, Li J, et al., medRxiv 2020:2020.03.02.20029975).

The changes in levels of macrophage-related cytokines observed herein may indicate that an immunomodulation of innate immunity and stromal cells, may potentially be part of the mechanism leading to clinical improvement. Additionally, in the present patient cohort elevated IL-6 levels (baseline median at 74.7 pg per milliliter), a higher median in the invasively ventilated versus the non-invasive group (157 vs. 69.2 pg per milliliter), and an overall decrease from the 74.7 pg per milliliter, to a median of 37.7 pg per milliliter three days after administration of maraviroc were observed. A recent publication reported high levels of IL-6 in 5 of 6 patients (range, 83 to 8175 pg per milliliter), which all decreased substantially three days after administration of the CCR5 inhibitor leronlimab (Akalin E, Azzi Y, Bartash R, et al., N Engl J Med 2020).

Patients with COVID-19 commonly have cytokine release syndrome (Moore JB, June CH., Science 2020;368:473-4), which is typically characterized by IL-6 increases and adverse clinical courses, and an association between high IL-6 levels and death was observed in a recent COVID-19 study (Zhou F, Yu T, Du R, et al., Lancet 2020;395:1054-62).

Understanding the complex influences that SARS-CoV-2 has on immune homeostasis, including differential protein expression and immune cell subset composition (Wen W, Su W, Tang H, et al., Cell Discov 2020;6:31), will be required to decipher the underlying mechanisms. Recent data also indicates that CCR5 inhibition via leronlimab may lead to a decrease in plasma viral load in severe COVID-19 cases (Patterson BK, Seethamraju H, Dhody K, et al., medRxiv preprint, not peer-reviewed 2020).

Taken together, remarkably positive clinical outcomes of severe COVID-19 cases treated with maraviroc was observed, likely due to an immunomodulatory effect on an overreacting immune system in acute COVID-19.

1. A method for treating and/or preventing pneumonia, sepsis, and/or coronavirus infection in a subject, comprising administering an effective amount of an immune modulator comprising at least one compound selected from
(i) a compound of formula I or a pharmaceutically acceptable salt or solvate thereof: 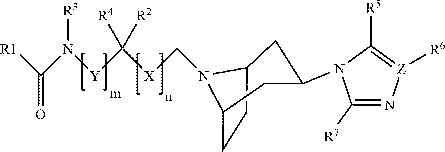
wherein
X and Y are independently selected from —CH₂— and —O—, preferably are —CH₂—;
Z is N or C, preferably is N;
m is an integer of from 0 to 3. preferably is 0;
n is an integer of from 1 to 3, preferably is 1;
R¹ is, optionally substituted, C₅-C₆ cycloalkyl or aryl;
R² is, optionally substituted, aryl or heteroaryl;
R³ and R⁴ are —H, or R³ and R⁴ together form a —CH₂—CH₂— bridge;
R⁵ and R⁷ are independently selected from alkyl, preferably methyl and isopropyl; and
in case Z is N, R⁶ is absent, or,
in case Z is C, R⁶ together with R⁵ forms a —CH═CH—CH═CH— bridge; and
(ii) a C—C chemokine receptor type 5 (CCR5) inhibitor .

2. The method of claim 1, wherein said compound of Formula 1 comprises
(i) m is 0;
(ii) X is — CH₂— and n is 1;
(iii) Z is N;
(iv) R¹ is 4,4-difluoro-cyclohexyl;
(v) R² is aryl, preferably phenyl;
(vi) R³ and R⁴ are —H; and
(vii) R⁵ is methyl and R⁷ is isopropyl .

3. The method of claim 1, wherein said immune modulator comprises a compound that has the activity of inhibiting CCR5, wherein said compound is selected from the list consisting of Maraviroc, Vicriviroc, Aplaviroc, ancriviroc, Cenicriviroc, TAK-779, E913, TAK-220, AZD5672, BMS-681. CCR5 antagonist 34. GSK2239633A. INCB009471, viral macrophage inflammatory protein-II, and CCL7.

4. The method of claim 1, wherein said immune modulator is a low-molecular weight compound with a molecular mass of at most 1 kDa.

5. The method of claim 1, wherein said immune modulator is selected from the list consisting of Maraviroc, Vicriviroc, and Aplaviroc.

6. The method of claim 1, wherein said treating and/or preventing further comprises administration of at least one antibiotic, preferably piperacillin, tazobactam, and/or caspofungin, and/or administration of hydroxychloroquine.

7. The method of claim 1, wherein said pneumonia is viral pneumonia, preferably coronavirus-associated pneumonia; wherein said sepsis is viral pneumonia-associated sepsis, preferably, is coronavirus pneumonia-associated sepsis, and/or wherein said coronavirus infection is coronavirus-associated pneumonia or coronavirus-associated sepsis.

8. The method of claim 1, wherein said coronavirus is severe acute respiratory syndrome coronavirus (SARS-CoV)-2, SARS-CoV-1, or Middle East respiratory syndrome coronavirus (MERS-CoV).

9. The method of claim 1, wherein said coronavirus is SARS-CoV-2.

10. The method of claim 1, wherein said subject is a human.

11. The method of claim 1, wherein said treating and/or preventing comprises reducing or inhibiting an immune response, preferably inhibiting IL-6 production, C-reactive protein production. IL-12 production, and/or reactive oxygen species production.

12. The method of claim 1, wherein said treating and/or preventing comprises reducing the hypo-inflammatory phase of sepsis, in particular comprises increasing monocyte, preferably macrophage counts, at inflammation sites.

13. The method of claim 1, wherein said treating and/or preventing comprises reducing antigen presentation by antigen presenting cells (APC).

14. A kit for practicing the method of claim 1, comprising an immune modulator as specified in claim 1 and a means of administration of said immune modulator to the lung.

15. A device for practicing the method of claim 1, comprising a device for administration of an immune modulator as specified in claim 1 to the lung, said device comprising an immune modulator as specified in claim 1.

16. The method of claim 1, wherein said immune modulator is Maraviroc and said coronavirus infection is a severe acute respiratory syndrome coronavirus (SARS-CoV)-2 infection.

17. The method of claim 1, wherein said immune modulator is administered in the range of a daily dose of 100 mg to 200 mg.

18. The method of claim 1, wherein said immune modulator is administered at a daily dose of 150 mg, preferably at two daily doses of 75 mg.

19. The method of claim 1, wherein said immune modulator has the activity of inhibiting CCR5.

20. The method of claim 2, wherein said immune modulator is Maraviroc.
